SEQUENCE LISTING (23 sequences)

SEQ ID NO: 1 - protein, 36 aa, Homo sapiens
YPIKPEAPGEDASPEELNRYYASLRHYLNLVTRQRY

SEQ ID NO: 2 - protein, 34 aa, Homo sapiens
  MISC_FEATURE (1)..(34): SEQ ID NO 1, residues 3-36
IKPEAPGEDASPEELNRYYASLRHYLNLVTRQRY

SEQ ID NO: 3 - protein, 40 aa, Artificial
  Synthetic PYY analogue
  MISC_FEATURE (1)..(34): SEQ ID NO 2, residues 1-34
IKPEAPGEDASPEELNRYYASLRHYLNLVTRQRYKKKKKK

SEQ ID NO: 4 - protein, 40 aa, Artificial
  Synthetic PYY analogue
  MISC_FEATURE (7)..(40): SEQ ID NO 2, residues 1-34
KKKKKKIKPEAPGEDASPEELNRYYASLRHYLNLVTRQRY

SEQ ID NO: 5 - protein, 46 aa, Artificial
  Synthetic PYY analogue
  MISC_FEATURE (7)..(40): SEQ ID NO 2, residues 1-34
KKKKKKIKPEAPGEDASPEELNRYYASLRHYLNLVTRQRYKKKKKK

SEQ ID NO: 6 - protein, 34 aa, Artificial
  Synthetic PYY analogue
  DISULFID (1)..(29)
  MISC_FEATURE (2)..(28): SEQ ID NO 2, residues 2-28
  MISC_FEATURE (30)..(34): SEQ ID NO 2, residues 30-34
CKPEAPGEDASPEELNRYYASLRHYLNLCTRQRY

SEQ ID NO: 7 - protein, 34 aa, Artificial
  Synthetic PYY analogue
  DISULFID (1)..(26)
  MISC_FEATURE (2)..(25): SEQ ID NO 2, residues 2-25
  MISC_FEATURE (27)..(34): SEQ ID NO 2, residues 27-34
CKPEAPGEDASPEELNRYYASLRHYCNLVTRQRY

SEQ ID NO: 8 - protein, 34 aa, Artificial
  Synthetic PYY analogue
  MISC_FEATURE (1)..(2): SEQ ID NO 2, residues 1-2
  DISULFID (3)..(24)
  MISC_FEATURE (4)..(23): SEQ ID NO 2, residues 4-23
  MISC_FEATURE (25)..(34): SEQ ID NO 2, residues 25-34
IKCEAPGEDASPEELNRYYASLRCYLNLVTRQRY

SEQ ID NO: 9 - protein, 34 aa, Artificial
  Synthetic PYY analogue
  MISC_FEATURE (1)..(4): SEQ ID NO 2, residues 1-4
  DISULFID (5)..(22)
  MISC_FEATURE (6)..(21): SEQ ID NO 2, residues 6-21
  MISC_FEATURE (23)..(34): SEQ ID NO 2, residues 23-34
IKPECPGEDASPEELNRYYASCRHYLNLVTRQRY

SEQ ID NO: 10 - protein, 33 aa, Artificial
  Synthetic PYY analogue
  MISC_FEATURE (1)..(8): SEQ ID NO 2, residues 1-8
  MISC_FEATURE (9)..(9)
  misc_feature (9)..(9): Xaa is 4-(2-Aminoethyl)6-dibenzofuranpropionic acid
  MISC_FEATURE (10)..(33): SEQ ID NO 2, residues 11-34
IKPEAPGEXSPEELNRYYASLRHYLNLVTRQRY

SEQ ID NO: 11 - protein, 34 aa, Artificial
  Synthetic PYY analogue
  MISC_FEATURE (1)..(8): SEQ ID NO 2, residues 1-8
  MISC_FEATURE (11)..(34): SEQ ID NO 2, residues 11-34
IKPEAPGEPPSPEELNRYYASLRHYLNLVTRQRY

SEQ ID NO: 12 - protein, 29 aa, Artificial
  Synthetic PYY analogue
  DISULFID (1)..(24)
  MISC_FEATURE (2)..(9): SEQ ID NO 2, residues 2-9
  MISC_FEATURE (10)..(23): SEQ ID NO 2, residues 20-28
  MISC_FEATURE (25)..(29): SEQ ID NO 2, residues 30-34
CKPEAPGEDLNRYYASLRHYLNLCTRQRY

SEQ ID NO: 13 - protein, 26 aa, Artificial
  Synthetic PYY analogue
  DISULFID (1)..(21)
  MISC_FEATURE (2)..(7): SEQ ID NO 2, residues 2-7
  MISC_FEATURE (8)..(20): SEQ ID NO 2, residues 16-28
  MISC_FEATURE (22)..(26): SEQ ID NO 2, residues 30-34
CKPEAPGNRYYASLRHYLNLCTRQRY

SEQ ID NO: 14 - protein, 29 aa, Artificial
  Synthetic PYY analogue
  DISULFID (1)..(2): SEQ ID NO 2, residues 1-2
  DISULFID (3)..(19)
  DISULFID (4)..(9): SEQ ID NO 2, residues 4-9
  DISULFID (10)..(18): SEQ ID NO 2, residues 15-23
  DISULFID (20)..(29): SEQ ID NO 2, residues 25-34
IKCEAPGEDLNRYYASLRCYLNLVTRQRY

SEQ ID NO: 15 - protein, 26 aa, Artificial
  Synthetic PYY analogue
  MISC_FEATURE (1)..(2): SEQ ID NO 2, residues 1-2
  DISULFID (3)..(16)
  MISC_FEATURE (4)..(7): SEQ ID NO 2, residues 4-7
  MISC_FEATURE (8)..(15): SEQ ID NO 2, residues 16-23
  MISC_FEATURE (17)..(26): SEQ ID NO 2, residues 25-34
IKCEAPGNRYYASLRCYLNLVTRQRY

SEQ ID NO: 16 - protein, 24 aa, Artificial
  Synthetic PYY analogue
  MISC_FEATURE (1)..(24): SEQ ID NO 2, residues 11-34
SPEELNRYYASLRHYLNLVTRQRY

SEQ ID NO: 17 - protein, 30 aa, Artificial
  Synthetic PYY analogue
  MISC_FEATURE (1)..(24): SEQ ID NO 2, residues 11-34
SPEELNRYYASLRHYLNLVTRQRYKKKKKK

SEQ ID NO: 18 - protein, 15 aa, Artificial
  Synthetic PYY analogue
  MISC_FEATURE (1)..(15): SEQ ID NO 2, residues 20-34
ASLRHYLNLVTRQRY

SEQ ID NO: 19 - protein, 21 aa, Artificial
  Synthetic PYY analogue
  MISC_FEATURE (1)..(15): SEQ ID NO 2, residues 20-34
ASLRHYLNLVTRQRYKKKKKK

SEQ ID NO: 20 - protein, 24 aa, Artificial
  Synthetic PYY analogue
  MISC_FEATURE (1)..(16): SEQ ID NO 2, residues 11-26
  MISC_FEATURE (17)..(17): Epsilon amino group in Lys-17 is bound to the C-terminus of SEQ ID NO 23 via a peptide bond
  MISC_FEATURE (18)..(24): SEQ ID NO 2, residues 28-34
SPEELNRYYASLRHYLKLVTRQRY

SEQ ID NO: 21 - protein, 15 aa, Artificial
  Synthetic PYY analogue
  MISC_FEATURE (1)..(7): SEQ ID NO 2, residues 20-26
  MISC_FEATURE (8)..(8): Epsilon amino group in Lys-8 is bound to the C-terminus of SEQ ID NO 23 via a peptide bond
  MISC_FEATURE (9)..(15): SEQ ID NO 2, residues 28-34
ASLRHYLKLVTRQRY

SEQ ID NO: 22 - protein, 21 aa, Artificial
  Synthetic PYY analogue
  MISC_FEATURE (1)..(5): SEQ ID NO 2, residues 1-5
  MISC_FEATURE (6)..(13): SEQ ID NO 2, residues 19-26
  MISC_FEATURE (14)..(14): Epsilon amino group in Lys-14 is bound to the C-terminus of SEQ ID NO 23 via a peptide bond
  MISC_FEATURE (15)..(21): SEQ ID NO 2, residues 28-34
IKPEAYASLRHYLKLVTRQRY

SEQ ID NO: 23 - protein, 5 aa, Artificial
  Synthetic PYY analogue
  MISC_FEATURE (1)..(5): SEQ ID NO 2, residues 1-5
IKPEA

(19) United States
(12) Patent Application Publication
(10) Pub. No.: US 2007/0244041 A1
(43) Pub. Date: Oct. 18, 2007

(54) Peptide Yy Analogues

(75) Inventors: Bjarne Larsen, Vanlose (DK); Lars B. Laurenborg Hansen, Herlev (DK)

Correspondence Address: EDWARDS ANGELL PALMER & DODGE LLP, P.O. BOX 55874, BOSTON, MA, 02205, US

(73) Assignee: Reoscience A/S, Rodovre (DK)

(21) Appl. No.: 10/590,634

(22) PCT Filed: Feb. 23, 2005
(86) PCT No.: PCT/EP05/01874

§ 371 Date: May 17, 2007

Related U.S. Application Data
(60) Provisional application No. 60/547,225, filed on Feb. 23, 2004.

(30) Foreign Application Priority Data
Feb. 23, 2004 (DK) PA 2004 00279

(51) Int. Cl. A61K 48/00; C07K 14/00

(52) U.S. Cl. 514/12; 530/350

(57) ABSTRACT

Analogues of the peptide PYY (1-36) are described in which the tertiary structure of the peptide is preserved and stabilised particularly to enhance binding and activation of the Y2 receptor by the use of cross links or rigid bends in the peptide to constrain conformationally the positions of the N-terminal part of the peptide sequence and amino acid 34. The analogues are useful in the control of food intake.

### FIELD OF THE INVENTION

The present invention relates to the field of appetite regulating therapy and therapy of diseases associated with appetite regulation. In particular, the present invention relates to novel enhanced analogues of peptide YY (3-36) and the use of these analogues in prevention and treatment of diseases associated with appetite regulation, such as obesity, anorexia, bulimia and cachexia.

### BACKGROUND OF THE INVENTION

Peptide YY (SEQ ID NO: 1) is a 36 amino-acid peptide belonging to the pancreatic polypeptide (PP) family of peptides also known as the PP-fold peptides because they share a common hairpin-like three-dimensional structure (Fuhlendorff et al., 1990, J Biol Chem 265:11706-12). Pancreatic polypeptide was the first of the PP-fold peptides to be discovered and received its name because it was isolated from insulin extracts (Kimmel et al., 1968, Endocrinology 83:1323-30). Peptide YY (PYY) and neuropeptide Y (NPY) were discovered later from intestinal and brain extracts respectively (Tatemoto et al., 1982, Nature 296:659-60, Tatemoto, 1982, Proc Natl Acad Sci USA 79:2514-8).

There are two main forms of endogenous PYY: PYY1-36 and PYY3-36 both of which can be found in the circulation (Grandt et al., 1994, Regul Pept 51:151-9, Grandt et al., 1994, Peptides 15:815-20). The enzyme dipeptidyl peptidase-IV hydrolyses PYY1-36 at the Pro2-Ile3 bond yielding PYY3-36 (SEQ ID NO: 2) (Medeiros and Turner, 1994, Endocrinology 134:2088-94). Peptide YY is synthesized by endocrine L-cells lining the gut and is released postprandially particularly following ingestion of fat (Adrian et al, 1985, Gastroenterology 89:1070-7). Plasma PYY levels increase within 15 minutes, are maximal at 90 minutes and are elevated for up to 6 hours following the ingestion of a meal (Adrian, et al., 1985, Gastroenterology 89:1070-7). In the fasted state PYY1-36 has been found to be the predominant form, whereas PYY3-36 predominates following a meal (Grandt, et al., 1994, Regul Pept 51:151-9, Grandt, et al., 1994, Peptides 15:815-20). In addition to intestinal L-cells PYY expression has also been demonstrated in a small population of neurons in the brainstem, suggesting that PYY could function as a neurotransmitter (Broome et al., 1985, Acta Physiol Scand 125:349-52).

To date five PP-fold receptors have been cloned and designated the Y1, Y2, Y4, Y5 and Y6 receptors (Berglund et al., 2003, Exp Biol Med (Maywood) 228:217-44). The existence of a Y3 NPY-preferring receptor has been suggested based on pharmacological studies, but the receptor remains to be cloned (Lee and Miller, 1998, Regul Pept 75-76:71-8). The lower case designation of the Y6 receptor is based on the fact that it encodes a truncated and presumably non-functional receptor in most mammals including humans (Michel et al., 1998, Pharmacol Rev 50:143-50). The functional Y-receptors are G-protein coupled receptors all coupling to inhibitory G-proteins (Gi) therefore inhibiting cAMP production (Berglund, et al., 2003, Exp Biol Med (Maywood) 228:217-44, Michel, et al., 1998, Pharmacol Rev 50:143-50).

The three PP-fold peptides, PYY, NPY and PP show different affinities to the Y-receptors. Whereas full length NPY and PYY show high affinity binding to Y1, Y2 and Y5 receptors, PYY3-36 and NPY3-36 show high selectivity for Y2 over Y1 receptors demonstrating the importance of the aminoterminal part of PP-fold peptides for Y1 receptor activation (Grandt et al., 1996, Regul Pept 67:33-7, Grandt et al., 1992, Biochem Biophys Res Commun 186:1299-306). In contrast, Y2 receptors are less strictly dependent on the amino-terminal portion, therefore permitting C-terminal truncated forms of PYY and NPY bind with almost equal affinity as the untruncated forms (Fuhlendorff, et al., 1990, J Biol Chem 265:11706-12). The Y4 subtype preferentially binds PP (Michel et al., 1998, Pharmacol Rev 50:143-50).

Peripheral administration of PYY produces a variety of primarily inhibitory effects on digestion. It has been shown that PYY injected into the systemic circulation inhibits gastric emptying and acid secretion, reduce stimulated pancreatic exocrine secretion and increase intestinal transit time (Pappas et al., 1985, Gastroenterology 89:1387-92, Pappas et al., 1986, Gastroenterology 91:1386-9, Adrian et al., 1985, Gastroenterology 89:494-9, Allen et al., 1984, Digestion 30:255-62). Inhibitory effects on digestive functions can also be elicited by injections of PYY into the hindbrain. Injection of PYY or PYY13-36 directly into the dorsal motor nucleus of the vagus can also inhibit gastric emptying (Martinez et al., 1998, Am J Physiol 274:G965-70, Chen and Rogers, 1997, Am J Physiol 273:R213-8, Browning and Travagli, 2003, J Physiol). These effects are presumably mediated by the Y2 receptor as PYY and PYY13-36 (the latter a Y2 selective agonist) equally effectively elicits the effects (Chen and Rogers, 1997, Am J Physiol 273:R213-8).

When PYY, NPY or PP are injected into the cerebral ventricles or into the hypothalamus (notably the paraventricular nucleus or lateral hypothalamic area) they all increse food intake (Campbell et al., 2003, J Neurosci 23:1487-97, Stanley et al., 1985, Peptides 6:1205-11). The stimulatory effects of NPY and PYY on food intake are believed to be mediated via activation of central Y1 and Y5 receptors (Berglund, et al., 2003, Exp Biol Med (Maywood) 228:217-44) whereas the orexigenic effects of PP presumably is caused by activation of Y4 receptors on neurons in the lateral hypothalamic area (Campbell et al., 2003, J Neurosci 23:1487-97). In contrast to the postsynaptic Y1, Y2 and Y5 receptors the prototypical response for the Y2 receptor is the presynaptic inhibition of neurotransmitter release (Wahlestedt et al., 1986, Regul Pept 13:307-18). This is consistent with the aforementioned predominantly inhibitory effects on vagal efferents. The Y2 agonist PYY13-36 applied onto vagal motor neurons inhibited the firing rate of approximately 50%, whereas only approximately 5% were activated (Chen and Rogers, 1997, Am J Physiol 273:R213-8).

Recently, an inhibitory role for post-prandially released PYY3-36 in appetite regulation was proposed (Batterham et al., 2002, Nature 418:650-4). It was shown that acute intraperitoneal (i.p.) injections of peptide YY (PYY₃₋₃₆) dose-dependently (30, 300 and 3000 μg/kg bw) inhibits 4 hour food intake and that chronic treatment (twice daily injections of 50 μg/kg bw of PYY₃₋₃₆) suppresses weight gain in rats (Batterham, et al., 2002, Nature 418:650-4). In the same study 90 min intravenous infusion of PYY3-36 to healthy human volunteers lead to a reduction in appetite and a reduced caloric intake for the following 12 hours (Batterham et al., 2002, Nature 418:650-4). The food inhibitory effect of peripherally administered PYY3-36 was recently shown to be present also in obese individuals (Batterham et al., 2003, N Engl J Med 349:941-8). The food inhibitory effect of PYY3-36 is presumably mediated by Y2 receptors, as mice lacking this receptor fail to reduce caloric intake when injected with PYY3-36 (30, 300 and 3000 μg/kg bw) (Batterham et al., 2002, Nature 418:650-4).

It has been suggested that peripherally administered PYY3-36 inhibits food via activation of presynaptic Y2 receptors on NPY neurons in the hypothalamic arcuate nucleus (Batterham et al., 2002, Nature 418:650-4). However, peripherally administered PP-fold peptides such as NPY and PYY gain access to the dorsal vagal complex (Whitcomb and Taylor, 1992, American Journal of the Medical Sciences 304:334-8), and vagal afferents terminating in the nucleus of the solitary tract are sensitive to several postprandially released gastrointestinal hormones (GLP-1, CCK). Thus, it is equally possible that Y2 receptors expressed in neurones of the dorsal vagal complex mediate the anorectic actions of peripheral PYY3-36.

Obesity, defined as an excess of body fat relative to lean body mass, is highly associated with important psychological and medical morbidities. Of these the most severe include Type II or non-insulin-dependent diabetes mellitus (NIDDM), hypertension, elevated blood lipids and coronary heart disease. Obesity, and especially upper body obesity, is the most common nutritional disorder of the world. Numerous studies indicate that lowering body weight dramatically reduces risk for chronic diseases, such as diabetes, hypertension, hyperlipidaemia, coronary heart disease, and musculo-skeletal diseases. For example, various measures of obesity, including, simple body weight, waist-to-hip ratios, and mesenteric fat depots, are strongly correlated with risk for non-insulin dependent diabetes (NIDDM), also known as type II diabetes. Obesity is also a risk factor for the group of metabolic derangements collectively named the metabolic syndrome or “Syndrome X”.

Current methods for promoting weight loss are not satisfactory. It is estimated that in the US alone approximately 33 billion USD is spent annually on weight reducing treatments, but considering that the prevalence of obesity continues to rise, the money spent appears largely futile.

The chronic nature of obesity, the worldwide epidemiological rise in the prevalence of obesity and the large number of associated diseases call for new methods and compositions such as pharmaceutical agents reducing caloric intake and hence promote weight-loss.

In addition to the vast number of people suffering from obesity as mentioned above, many individuals are suffering from eating disorders, which are serious and life-threatening conditions, wherein gaining body weight and enhancing body fat are essential parts of treating said disorders. Such disorders are i.a. anorexia, bulimia and cachexia. The latter disorder is a well-known devastating complication of cancer, where many patients suffer from malnutrition. Cachexia occurs in more than two thirds of patients who die with advanced cancer and is the single most common documented cause of death in cancer (Nelson, K. A., Journal of Clinical Oncology, Vol. 12, No 1(January), 1994, pp 213-225).

Cachexia related to cancer is a syndrome characterised by host tissue wasting and anorexia amongst other symptoms (Albrecht, J. T., Paraneoplastic Syndromes, Vol. 10, No 4, 1996 pp 791-800).

### OBJECT OF THE INVENTION

It is an object of the present invention to provide improvements in the treatment of appetite regulating diseases, such as obesity and eating disorders, such as anorexia and bulimia as well as cancer related cachexia to provide agents effective in the treatment of conditions characterized by deposition of too little/reduced or excess body fat and excess or too low/reduced energy consumption.

### SUMMARY OF THE INVENTION

The present invention is based on the finding that the C-terminus of naturally occurring PYY is essential for its appetite regulating properties, whereas the binding to the relevant receptors can be contributed to other parts of the molecule.

The present inventors herein present a series of PYY analogues that have been devised to preserve or enhance the appetite regulating properties of PYY. Inter alia, the invention provides Y2 agonists in the form of PYY (3-36) analogues.

Thus, in one aspect, the present invention relates to a peptide, which is a sequence variant and a functional and/or structural mimic of peptide YY, especially a Y2 agonist, said peptide comprising at least one modification of the amino acid sequence set forth in SEQ ID NO: 2 (h-PYY 3-36), wherein said peptide
(a) includes a modification that conformationally constrains the relative position of the N-terminal one of the amino acids of SEQ ID NO 2 present in the peptide and amino acid 34 of SEQ ID NO: 2; and/or
(b) includes a branched amino acid sequence resulting in 2 free N-terminal amino acids; and/or
(c) includes N-terminal and/or C-terminal addition of a net basic amino acid sequence;
(d) optionally further includes deletion of amino acids 1-5 of SEQ ID NO: 2; and/or
(e) includes deletion of any one or more of amino acid residues 8-15 of SEQ ID NO: 2 without deletion of all of amino acids 1-7 of SEQ ID NO 2; and/or
(f) includes deletion of amino acids 6 and 7 of SEQ ID NO: 2 without deletion of all of amino acids 1-5 of SEQ ID NO 2; and/or
(g) includes deletion of amino acids 16-19 of SEQ ID NO: 2 without deletion of all of amino acids 1-15 of SEQ ID NO 2; and/or
(h) includes two cross linkable, optionally modified or protected, Cys amino acid substitutions;
   wherein said peptide further comprises at most 6 substitutions in the amino acid sequence set forth in SEQ ID NO: 2, each of which is a structure and/or functionality preserving substitution. It should be noted that SEQ ID NO 2 sets out the sequence of h-PYY (3-36) so that amino acid number 1 in SEQ ID NO 2 is the amino acid referred to as 3 in the nomenclature PYY (3-36) and the sequence ends at amino acid number 34 with the amino acid signified by the number 36 in the nomenclature PYY (3-36).

Optionally, it may be required that only one of features (a)-(h) is present or that only one of features (a), (b), (c) or (h) is present, optionally in combination with one or more of the remaining features.

In an alternative aspect, the invention includes a peptide, which is a sequence variant and a functional mimic of peptide YY, said peptide comprising at least one modification of the amino acid sequence set forth in SEQ ID NO: 2, wherein said peptide
includes a modification that conformationally constrains the relative position of amino acids 1 and 34 of SEQ ID NO: 2; and/or
includes N-terminal and/or C-terminal addition of a net basic amino acid sequence; and/or
includes deletion of any one of amino acid residues 8-15 of SEQ ID NO: 2; and/or
includes deletion of amino acids 1-5 of SEQ ID NO: 2; and/or
includes deletion of amino acids 6 and 7 of SEQ ID NO: 2; and/or
includes deletion of amino acids 16-19 of SEQ ID NO: 2; and/or
includes a branched amino acid sequence resulting in 2 free N-terminal amino acids;
   wherein said peptide further comprises at most 6 structure and/or functionality preserving substitutions in the amino acid sequence set forth in SEQ ID NO: 2.

The present invention further relates to peptides of formula I, discussed below, as well as to methods of preparing the peptides. Pharmaceutical compositions comprising the peptides are also part of the invention as are methods of preventing and treating conditions that are characterized by excess body fat deposition.

### BRIEF DESCRIPTION OF THE DRAWING

FIG. 1: Shows in outline an in vivo experimental setup for assessing efficacy of PYY analogues. For 7 weeks mice are kept 5 per cage and fed a high-fat (HF) diet. At the beginning of week 8 (the arrow marked “1” in the figure), the animals are kept 1 per cage; body-weight and food intake is monitored bi-weekly from this point. On day 0 (arrow marked “A” in the figure) animals have Alzet osmotic pumps (model 2004) implanted. Following the operation, mice are allowed to recover, then transferred back to their cages. For the following 26 days, food intake and body-weight is monitored bi-weekly until termination of the experiment.

FIG. 2: Shows a graphical representation of the suppression of body weight gain upon feeding rats a Y2 agonist as per Example 5.

### DETAILED DISCLOSURE OF THE INVENTION

In the following, a number of definitions will be presented for the purposes of understanding the present invention:

The term “peptide” herein designates any molecule comprising a chain of amino acids that are linked by means of a peptide bond. The term thus embraces molecules that include moieties that are not amino acids, but it will be understood that the peptides presented in the present specification and claims predominantly consists of amino acids that are joined by means of peptide bonds.

The term “peptide YY” or PYY denotes the peptide having the sequence set forth in SEQ ID NO: 2, i.e. PYY-3-36, unless otherwise indicated.

The term “amino acid” refers to a molecule having the general formula R—C(NH₂)—COOH which is capable of forming a peptide bond with another molecule having the same general formula. The term embraces both L and D amino acids.

A “naturally occurring amino acid” is in the present context one of the 20 amino acids Group Ala (A), Cys (C), Ser (S), Thr (T), Asp (D), Glu (E), Asn (N), Gln (Q), His (H), Arg (R), Lys (K), Ile (I), Leu (L), Met (M), Val (V), Phe (F), Tyr (Y), Trp (W), Gly (G), and Pro (P).

Normally, these are L-amino acids, but the present invention also allows for the use of these amino acids in their D-form.

“Unusual amino acids” refer to amino acids that are either rare in nature or purely synthetic. Unusual amino acids used in this invention can (as the naturally occurring) be synthesized by standard methods familiar to those skilled in the art (“The Peptides: Analysis, Synthesis, Biology, Vol. 5, pp. 342-449, Academic Press, New York (1981)). N-Alkyl amino acids can be prepared using procedures described in previously (Cheung et al., (1977) Can. J. Chem. 55, 906; Freidinger et al., (1982) J. Org. Chem. 48, 77 (1982)), which are incorporated herein by reference.

A “structure preserving substitution” refers to the substitution of an amino acid residue with another amino acid residue having similar characteristics or properties including charge, hydrophobicity, etc., such that the overall structure of the substituted product does not change significantly when compared to the unsubstituted PYY.

A “functionality preserving substitution” refers to the substitution of an amino acid residue with another amino acid residue having similar characteristics or properties including size, charge, hydrophobicity, etc., such that the overall functionality of the substituted product does not change significantly when compared to the unsubstituted PYY.

Some functionality preserving or structure preserving substitutions are those known as conservative substitutions, i.e. substitutions with naturally occurring amino acids that, based on evolutionary studies, are known to only introduce minor functional changes in proteins where they occur.

In the context of the present invention, amino acids belonging to each of the following groups can be interchanged freely within the same group when performing a substitution:
Group 1: Ala (A), Cys (C), Ser (S), and Thr (T);
Group 2: Asp (D) and Glu (E);
Group 3: Asn (N), Gln (Q) and His (H);
Group 4: Arg, Lys, Ornithin, Dab (1,4 diaminobutyric acid), and Dapa (1,3 diaminopropionic acid);
Group 5: Ile (I), Leu (L), Met (M), and Val (V);
Group 6: Phe (F), Tyr (Y), and Trp (W);
Group 7: Gly (G) and Pro (P).

It should be noted, that conservative substitutions “allowed” according to the PAM (“Point Accepted Mutations”) or Blosum matrices (“BLOCKS SUbstitution Matrix, Henikoff and Henikoff, 1992; PNAS 89:10915-10919) are also regarded as functionality-conserving substitutions within the meaning of the present invention.

A “rigid bend” in a peptide is in the present context a conformational constraint in the amino acid chain. In nature, it is known that proline residues introduce a fixed angle in an amino acid chain, because the amino group that is part of the peptide bond also is parts of a ring structure, meaning that there is no free rotation. Similarly, amino acids having “bulky” or charged side groups may be sterically hindered from attaining all conformations if neighbouring amino acid residues are somehow capable of interacting with these residues.

A “multimer” denotes a molecule that includes at least two identical peptides of the present invention, either as a linear repeat of the same peptide sequence where the peptides are joined end-to-end, or in the form of covalently or non-covalently linked copies of peptides of the invention that are not joined end-to-end. This may include aggregation via non-covalent “weak bonds” or interpeptide disulphide or amide bonds. According to the invention, dimers are especially attractive multimer versions of the peptides of the present invention.

A “structural mimic” of peptide YY is a peptide of the invention, which has substantially the same or an enhanced IC50 value when compared to peptide YY when measured as binding to receptor Y2 in the assay set forth in example 2 or binding to receptor Y5 in the assay set forth in example 3. This means that a structural mimic must exhibit an appetite-reducing or appetite-enhancing effect in vivo in humans or in an appropriate animal model, where peptide YY would also be effective.

A “functional mimic” of peptide YY is a peptide of the invention, which has substantially the same or an enhanced EC50 value when compared to peptide YY when measured in the efficacy assay set forth in example 2. This means that a functional mimic must exhibit an appetite-reducing effect in vivo in humans or in an appropriate animal model, where peptide YY would also be effective.

A compound shall be considered to be ‘conformationally constrained’ to define the relative positions of amino acids 1 and 34 of SEQ ID NO 2 at least if it has a cross-link between an amino acid in the sector defined by amino acids 1-6 (preferably 1-5) of SEQ ID NO 2 and an amino acid in the sector defined by amino acids 12-30 (preferably 22-29) of SEQ ID NO 2. Also, a compound shall be considered to be ‘conformationally constrained’ to define the relative positions of amino acids 1 and 34 of SEQ ID NO 2 at least if it has substitutions of amino acids imposing a rigid bend substituted or added into SEQ ID NO 2 in the sector defined by amino acids 7-11 (preferably 9-10) of SEQ ID NO 2. A rigid bend shall be present at least if said substitution or addition provides in this region a dipeptide moiety A-B of the formula Gly-Gly, Pro-Gly, Gly-Pro, Sar-Sar, Sar-Hyp, Hyp-Sar, Pro-Sar, Sar-Pro, Pro-Hyp, Pro-Pro, Hyp-Pro, and Hyp-Hyp, where Pro and Hyp independently may be an L or D form, where the ring structure of Pro and Hyp is optionally substituted with halogen, nitro, methyl, amino, or phenyl, Hyp represents 3-hydroxyproline or 4-hydroxyproline, Sar represents sarcosine, or one or both of the amino acid residues of A-B is a Sar, or an N-cyclohexylglycine residue, or A and B each independently represents a group of the formula II 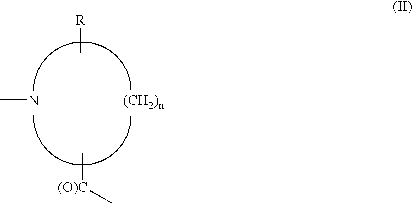
wherein n is an integer having the value 3, 4, or 5, and R represents an optional substituent, preferably selected from the group consisting of halogen, phenyl, hydroxy, NH₂, and C(1-6)alkyl optionally substituted with halogen, or
A-B designates the formula IIa 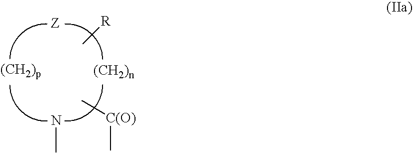
   wherein n is an integer having the value 0, 1, 2, and 3, p is an integer having the value 0, 1, 2, and 3, Z represents O or S, and R represents an optional substituent, preferably selected from the group consisting of halogen, phenyl, hydroxy, NH₂, and C(1-6)alkyl, or A and B independently represents an amino acid residue having a saturated carbocyclic structure of 4, 5 or 6 members and where in said carbocyclic structure further comprises one or more heteroatoms,

### DESCRIPTION OF PREFERRED EMBODIMENTS OF THE INVENTION

The present invention discloses a number of analogues of Peptide YY that all aim at preserving the C-terminus of the peptide having SEQ ID NO: 2. A number of other analogues of the invention aim at also preserving and/or stabilising the hairpin-like structure of peptide YY, since it is believed that this structure has big impact on the receptor interaction. As mentioned above, this is done by modifying SEQ ID NO: 2 so that a conformational constraint is introduced which fixes the relative 3D positions of amino acids 1 and 34 of SEQ ID NO: 2 (or which would do so, if amino acid no. 1 in SEQ ID NO: 2 was present in the analogue—some of the analogues includes deletions of the N-terminal part of Peptide YY but include modifications that would, in an intact 34 amino acid long peptide, constrain amino acid no. 1 and 34 relative to each other).

Such modifications that conformationally constrain the relative position of amino acids 1 and 34 of SEQ ID NO: 2 may according to the invention be selected from the group consisting of introduction of a disulfide bridge, introduction of a rigid bend (e.g. by introducing two proline residues, cf. below) , especially involving positions corresponding to residues 9 and 10 in SEQ ID NO: 2, and introduction of at least one stabilising amide bond between amino acid side chains.

It is also possible to include terminal (N-terminal and/or C-terminal) additions of amino acids that serve to stabilise the analogues against degradation. According to the invention, this is typically done by adding a net basic amino acid sequence to either or both termini.

Further, parts of the amino acid sequence of Peptide YY may be deleted in the analogues of the invention; as mentioned above, it is noted that the appetite regulating properties of Peptide YY is highly dependent on an intact C-terminus, whereas the function of other parts of the molecule seems to be facilitation of receptor binding—this binding, however, is not in itself enough to bring about the appetite regulating effects of Peptide YY, and as part of the present invention, it is contemplated to provide deletion variants, such as those variants that include deletion of any one of amino acid residues 8-15 of SEQ ID NO: 2 and/or deletion of amino acids 1-5 of SEQ ID NO: 2 and/or include deletion of amino acids 6 and 7 of SEQ ID NO: 2; and/or include deletion of amino acids 16-19 of SEQ ID NO: 2.

Finally, a specific subset of peptide YY analogues of the present invention are designed to fixate the N- and C-terminals, i.e. it is within the scope of the invention to provide analogues having various distances in space between the N-terminal part of the peptide and the C-terminal part of the peptide. This is achieved by including a branched amino acid sequence resulting in 2 free N-terminals

Another part of the present invention relates to a peptide (which may be a peptide as described above) of formula I
R¹-X-Y-Z-A²²-A²³-A²⁴-A²⁵-A²⁶-A²⁷-A²⁸-A²⁹-A³⁰-A³¹-A³²-A³³-A³⁴-A³⁵-A³⁶-R² (I)
wherein
A²² is Ala or a structure and/or functionality preserving substitution thereof;
A²³ is Ser or a structure and/or functionality preserving substitution thereof;
A²⁴ is Leu or a structure and/or functionality preserving substitution thereof, His or Cys;
A²⁵ is Arg or a structure and/or functionality preserving substitution thereof;
A²⁶ is Leu or a structure and/or functionality preserving substitution thereof, His or Cys;
A²⁷ is Tyr or a structure and/or functionality preserving substitution thereof;
A²⁸ is Leu or a structure and/or functionality preserving substitution thereof, or Cys;
A²⁹ is Asn or a structure and/or functionality preserving substitution thereof, or Lys which is optionally coupled to an amino acid sequence via a peptide bond at the ε-amino group;
A³⁰ is Leu or a structure and/or functionality preserving substitution thereof;
A³¹ is Val or a structure and/or functionality preserving substitution thereof, or Cys;
A³² is Thr or a structure and/or functionality preserving substitution thereof;
A³³ is Arg or a structure and/or functionality preserving substitution thereof;
A³⁴ is Gln or a structure and/or functionality preserving substitution thereof;
A³⁵ is Arg or a structure and/or functionality preserving substitution thereof; and
A³⁶ is Tyr or a structure and/or functionality preserving substitution thereof;

Z is a peptide of formula
A¹³-A¹⁴-A¹⁵-A¹⁶-A¹⁷-A¹⁸-A¹⁹-A²⁰-A²¹
which is absent or wherein,
A¹³ is Ser or a structure and/or functionality preserving substitution thereof or absent;
A¹⁴ is Pro or a structure and/or functionality preserving substitution thereof or absent;
A¹⁵ is Glu or a structure and/or functionality preserving substitution thereof or absent;
A¹⁶ is Glu or a structure and/or functionality preserving substitution thereof or absent;
A¹⁷ is Leu or a structure and/or functionality preserving substitution thereof or absent;
A¹⁸ is Asn or a structure and/or functionality preserving substitution thereof;
A¹⁹ is Arg or a structure and/or functionality preserving substitution thereof;
A²⁰ is Tyr or a structure and/or functionality preserving substitution thereof; and
A²¹ is Tyr or a structure and/or functionality preserving substitution thereof;

Y is a peptide of formula
A⁸-A⁹-A¹⁰-A-B
which is absent or wherein
A⁸ is Pro or a structure and/or functionality preserving substitution thereof;
A⁹ is Gly or a structure and/or functionality preserving substitution thereof;
A¹⁰ is Glu or a structure and/or functionality preserving substitution thereof, or absent; and
A-B designates a dipeptide A¹¹-A¹² selected from the group consisting of Gly-Gly, Pro-Gly, Gly-Pro, Sar-Sar, Sar-Hyp, Hyp-Sar, Pro-Sar, Sar-Pro, Pro-Hyp, Pro-Pro, Hyp-Pro, and Hyp-Hyp, where Pro and Hyp independently may be an L or D form, where the ring structure of Pro and Hyp is optionally substituted with halogen, nitro, methyl, amino, or phenyl, Hyp represents 3-hydroxyproline or 4-hydroxyproline, Sar represents sarcosine, or one or both of the amino acid residues of A-B is a Sar, or an N-cyclohexylglycine residue, or

A and B each independently represent a group of the formula II 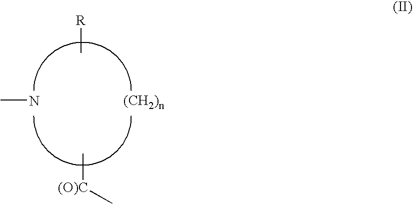
wherein n is an integer having the value 3, 4, or 5, and R represents an optional substituent, preferably selected from the group consisting of halogen, phenyl, hydroxy, NH₂, and C(1-6)alkyl optionally substituted with halogen, or
A-B designates the formula IIa 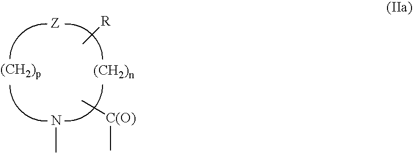
   wherein n is an integer having the value 0, 1, 2, and 3, p is an integer having the value 0, 1, 2, and 3, Z represents 0 or S, and R represents an optional substituent, preferably selected from the group consisting of halogen, phenyl, hydroxy, NH₂, and C(1-6)alkyl, or
A and B independently represents an amino acid residue having a saturated carbocyclic structure of 4, 5 or 6 members and where in said carbocyclic structure further comprises one or more heteroatoms, or
A is absent, Asp or a structure and/or functionality preserving substitution thereof and B is absent, Ala or a structure and/or functionality preserving substitution thereof;

X is a peptide of formula
A³-A⁴-A⁵-A⁶-A⁷
which is absent or wherein
A³ is Ile or a structure and/or functionality preserving substitution thereof, or Cys;
A⁴ is Lys or a structure and/or functionality preserving substitution thereof;
A⁵ is Pro or a structure and/or functionality preserving substitution thereof, or Cys;
A⁶ is Glu or a structure and/or functionality preserving substitution thereof; and
A⁷ is Ala or a structure and/or functionality preserving substitution thereof, or Cys;
R¹ is absent or an amino acid sequence; and
R² is absent or an amino acid sequence;
   wherein said peptide comprises at most one disulfide bridge selected from Cys³-S—S-Cys³¹ (e.g. SEQ ID NOS 6, 12 AND 13), Cys³-S—S-Cys²⁸ (e.g. SEQ ID NO 7), Cys⁵-S—S-Cys²⁶ (e.g. SEQ ID NOS 8, 14 AND 15), and Cys⁷-S—S-Cys²⁴ (e.g. SEQ ID NO 9),
   wherein the number of structure and/or functionality preserving substitutions does not exceed 6;
wherein the C-terminal amino exposes a free carboxylic acid group or an amide group; and
wherein the peptide does not consist of any of the amino acid sequences set forth in SEQ ID NO: 1 and SEQ ID NO: 2,
   or a multimer and/or pharmaceutically acceptable salt thereof.

Normally the number of functionality preserving substitutions in formula I will be kept at a minimum, meaning that the peptide will include 5, 4, 3, 2, 1 or even 0 structure and/or functionality preserving substitutions.

As will appear, all peptides of formula I include the substituents A²²-A³⁶, i.e. corresponding to the part of peptide YY (SEQ ID NO: 2, residues 20-34) which are believed to be essential for the appetite regulating effects exerted by this peptide.

In one embodiment of the invention it is preferred that a peptide according to the invention binds to receptor Y2. By this is meant a specific, significant binding that can be clearly distinguished from the binding by some irrelevant substance to the receptor, e.g. the binding by serum proteins. It is further preferred that a peptide of the invention binds with higher affinity to receptor Y2 than to receptor Y1, since the appetite-regulating effects of peptide YY have been demonstrated to be a consequence of interaction with receptor Y2, whereas the binding to receptor Y1 seems of limited relevance for the purposes of the present invention, cf. the discussion of receptor affinities in the background of the invention section.

In another embodiment of the invention it is preferred that a peptide according to the invention binds to receptor Y5. By this is meant a specific, significant binding that can be clearly distinguished from the binding by some irrelevant substance to the receptor, e.g. the binding by serum proteins. In this particular embodiment it is further preferred that a peptide of the invention binds with higher affinity to receptor Y5 than to receptor Y1.It is especially attractive that a peptide of the invention binds specifically with the Y2 receptor or the Y5 receptor so that the ratio between affinities for receptor Y2 or receptor Y5 and receptor Y1 is at least 10, but higher ratios are preferred and contemplated, such as at least 20, at least 30, at least 40, at least 50, at least 60, at least 70, at least 80, at least 90, and at least 100.

However, some residual binding to the Y1 receptor are expected for some specific peptide analogues of the invention, especially those where a large proportion of the N-terminus has been preserved. Therefore, a peptide of the invention may have some binding to the Y1 receptor, meaning that ratio between affinities for receptor Y2 or receptor Y5 and receptor Y1 is at most 200, such as at most 190, at most 180, at most 170, at most 160, at most 150, at most 140, at most 130, at most 120, and at most 110. It will be understood however, that the affinity to the Y1 and Y2 and Y5 receptors are not the only feature that will provide preferred peptides of the invention.

As mentioned above, the ultimately interesting parameter is the ability of the peptide of the invention to regulate appetite and thereby prove to be a feasible candidate for an anti-obesity drug or an appetite-enhancing drug. In other words, also peptides having formula I set forth above must preferably be structural and/or functional mimics of peptide YY, i.e. of the peptide having the sequence set forth in SEQ ID NO: 2. Structural mimicry of the peptides of the invention is, according to the present invention, preliminarily gauged in the 2 receptor binding assays that are described in example 2 and example 3. Preferred peptides of the invention in any or both of these two assays exhibit an IC50 value which is at least 40% of that of peptide YY, such as at least 50%, at least 60%, at least 70%, at least 80%, at least 90%, at least 100%, at least 110%, at least 120%, at least 130%, at least 140%, and at least 150% of the IC50 value of the peptide having the amino acid sequence set forth in SEQ ID NO: 2.

Functional mimicry of the peptides of the invention is, according to the present invention, preliminarily gauged in the efficacy assay described in example 2. Preferred peptides of the invention in these two assays exhibit an EC50 value which is at least 40% of that of peptide YY, such as at least 50%, at least 60%, at least 70%, at least 80%, at least 90%, at least 100%, at least 110%, at least 120%, at least 130%, at least 140%, and at least 150% of the EC50 value of the peptide having the amino acid sequence set forth in SEQ ID NO: 2. In one embodiment the preferred peptides of the invention exhibit an EC50<1 nM in the efficacy assay set forth in Example 2 and/or exhibits an IC50<1 nM in the Y2-binding assay set forth in Example 2 and/or exhibits an IC50<1 nm in the Y5-binding assay set forth in Example 3.

Preferred Embodiments of Peptides Having Formula I

In preferred variants of formula I, A²⁹ is Lys. Advantageously, Lys²⁹ is in these cases coupled to an amino acid sequence via a peptide bond at the ε-amino group, preferably to a peptide having the amino acid sequence set forth in SEQ ID NO: 23. However, this peptide coupled to Lys²⁹ may also be a truncate of SEQ In NO: 23, where one or two of the C-terminal amino acids in SEQ ID NO: 23 has been deleted to leave only the 3-4 N-terminal amino acids thereof.

In preferred embodiments, especially for Y2 agonists, the peptide of formula I includes the disulfide bridge Cys³-S—S-Cys³¹ and/or the disulfide bridge Cys³-S—S-Cys²⁸ and/or the disulfide bridge Cys⁵-S—S-Cys²⁶ and/or the disulfide bridge Cys⁷-S—S-Cys²⁴. In some preferred embodiments, at most one of A²⁴, A²⁶, A²⁸, and A³¹ in formula I is Cys, meaning that at most one of the stabilising disulfide bridges can be formed between the N- and C-terminal parts of the peptide of the invention.

In the peptide having formula I, preferred embodiments include substituent X having the amino acid sequence set forth in SEQ ID NO: 23. However, in a number of embodiments, X is absent.

In substituent Y, A and B, may independently be selected from the group consisting of N- and C(O)-radicals of the following compounds:
D/L-azetidin-3-carboxylic acid,
D/L-azetidin-2-carboxylic acid,
D/L-Indolin-2-carboxylic acid,
D/L-1,3-dihydro-isoindol-1-carboxylic acid,
D/L-thiazolidin-4-carboxylic acid,
D/L-pipecolinic acid,
D/L-nipecotinic acid,
isonipecotinic acid,
L/D-2-carboxymorpholin,
L/D-1,2,3,4-tetrahydroquinolin-3-carboxylic acid,
L/D-1,2,3,4-tetrahydroquinolin-3-carboxylic acid, and
4-carboxy-4-phenyl-piperidin.

In other, especially preferred embodiments, A-B designates 4-(2-aminoethyl)-6-dibenzofuranpropionic acid.

A-B in some embodiments preferably constitutes a dipeptide; it is especially preferred that A and B both designate Pro or a derivative thereof, and it is contemplated that Pro or its derivative, independently, is an L or D form. The derivative of Prolin typically has one or more substituents in the 3, 4 or 5 position, said substituents preferably being selected from hydroxy, amino and phenyl.

In a further embodiment, A and B independently represents an amino acid residue having a saturated carbocyclic structure of 4, 5 or 6 members, wherein said carbocyclic structure further comprises one or more heteroatoms selected from the group consisting of N, O and S. Said amino acids include L and D forms, natural and unnatural amino acids and derivatives thereof, such as a prolin residue having one or more substituents in the 3, 4 or 5 position, said substituents being preferably selected from hydroxy, amino or phenyl; and N-substituted amino acids, such as Sarcosin, N-cyclohexylglycine, and N-phenylglycine.

The peptide having formula I include certain embodiments where B, A¹³, A¹⁴, A¹⁵, and A¹⁶ are absent. In some of these embodiments A¹⁰, A, and A¹⁷ may be present, but in other A¹⁰, A, and A¹⁷ are also absent, meaning that A¹⁰, A, B, A³, A¹⁴, A¹⁵, A¹⁶, and A¹⁷ are absent. In these embodiments (i.e. both those where A¹⁰, A, and A¹⁷ are present and absent) it is preferred that A⁸, A⁹, A¹⁸, A¹⁹, A²⁰, and A²¹ are present.

To summarize, in a number of embodiments of formula I, substituents X, Y and Z may be present or absent according to the following scheme:

| X | Y | Z |
|---|---|---|
| Present | Present | Present |
| Absent | Present | Present |
| Absent | Absent | Present |
| Absent | Present | Absent |
| Absent | Absent | Absent |
| Present | Absent | Present |
| Present | Present | Absent |
| Present | Absent | Absent |

R¹ in Formula I preferably designates an amino acid sequence having between 4 and 20 amino acid residues, and it is especially preferred that the amino acid sequence has 6 amino acid residues. In these embodiments, the amino acid residues constituting R¹, are basic. R¹ is in this case often selected from Lys, Arg, His, and Orn. In the most preferred embodiment in this context, R¹ consists of six Lys residues.

R² in Formula I preferably designates an amino acid sequence having between 4 and 20 amino acid residues, and it is especially preferred that the amino acid sequence has 6 amino acid residues. In these embodiments, the amino acid residues constituting R², are basic. R² is in this case often selected from Lys, Arg, His, and Orn. In the most preferred embodiment in this context, R² consists of six Lys residues.

In some embodiments, both R¹ and R² in Formula I preferably designate an amino acid sequence having between 4 and 20 amino acid residues as detailed in the two foregoing paragraphs.

In an especially preferred embodiment R¹ designates the result of acylation of X with an optionally substituted straight, branched, saturated, unsaturated, or aromatic C(1-22)carboxylic acid where the substitutent is selected from hydroxy, halogen, C(1-6)alkyl, nitro or cyano and may be situated on the carbon chain or the aromatic moiety; preferred C(1-22)carboxylic acids are C(1-7)carboxylic acids selected from the group consisting of acetic acid, propionic acid, butyric acid and isomers thereof, and benzoic acid. The C(1-6)alkyl is chosen amongst methyl, ethyl, propyl, isopropyl, butyl, 1-methyl-propyl, 2-methyl-propyl, 1,1-dimethyl-ethyl, pentyl, 1-methyl-butyl, 2-methyl-butyl, 3-methyl-butyl, 1-ethyl-propyl, 1,1-methyl-propyl, 2,2-methyl-propyl, 1,2-methyl-propyl, hexyl, 1-methyl-pentyl, 2-methyl-pentyl, 3-methyl-pentyl, 4-methyl-pentyl, 1-ethyl-butyl, 2-ethyl-butyl, 1,1-methyl-butyl, 2,2-methyl-butyl, 1,2-methyl-butyl, 1,3-methyl-butyl, 2,3-methyl-butyl, 3,3-methyl-butyl, 1,1,2-trimethyl-propyl, 1-methyl-1-ethyl-propyl, 1-ethyl-2-methyl-propyl, and 1-methylethyl-propyl.

The most preferred peptides of the present invention are: SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, and SEQ ID NO: 22.

The following analogues have been synthesised:

| | | Preferred |
|---|---|---|
| | | as Y2 |
| Compound | Comment | agonists |
| h-PYY(3-36)-NH₂ | Conformationally | y |
| Pro-Pro 11-12 | constrained by the | |
| | Pro Pro linkage | |
| h-PYY(3-36)-NH₂ | Conformationally | y |
| Dbf 11-12 | constrained | |
| | by Dbf 11-12 | |
| K₆-h-PYY(3-36)-NH₂ | Includes an added | |
| | N-terminal net basic | |
| | amino acid chain | |
| h-PYY(22-36)-K₆—NH₂ | Includes an added | |
| | C-terminal net basic | |
| | amino acid chain | |
| h-PYY(13-36)-K₆—NH₂ | Includes an added | |
| | C-terminal net basic | |
| | amino acid chain | |
| IKPE-h-PYY-(13-36)- | Branched to include two | |
| K29(H-IKPEA)-NH₂ | N-terminal amino acids | |
| h-PYY(3-36)-K₆—NH₂ | Includes an added | |
| | C-terminal net basic | |
| | amino acid chain | |
| h-PYY-(13-36)- | Branched to include two | |
| K29(H-IKPEA)-NH₂ | N-terminal amino acids | |
| DesAsp-h-PYY(3-36)-NH₂ | Deletion of one of | y |
| | amino acids 8-15 of | |
| | SEQ ID NO 2 | |
| Des12-16-h-PYY(3- | Deletion of amino acids | y |
| 36)-NH₂ 3,31-bis | 10-14 of SEQ ID NO 2 | |
| Cys(Acm) | | |
| Des12-16-h-PYY(3- | Deletion of amino acids | y |
| 36)-NH₂ (C3-C31) | 10-14 of SEQ ID NO 2 | |
| | and Cys substitution | |
| | and cross linking of | |
| | SEQ ID NO 2 | |
| | amino acids 1 and 29 | |
| Des10-17-h-PYY(3- | Deletion of amino | y |
| 36)-NH₂ 3,31-bis | acids 8-15 of SEQ | |
| Cys(Acm) | ID NO 2 and Cys(Acm) | |
| | substitution of SEQ | |
| | ID NO 2 amino acids | |
| | 1 and 29 | |
| Des10-17-h-PYY(3- | | |
| 36)-NH₂ (C3-C31) | | |
| h-PYY(3-36)-NH₂ | Cross linkable | y |
| 3,31-bis Cys(Acm) | protected Cys amino | |
| | acid substitutions | |
| h-PYY-(3-36)-NH₂ | Cross linked Cys | y |
| (C3-C31) | amino acid | |
| | substitutions | |
| h-PYY(3-36)-NH₂ | Cross linkable | y |
| 7,24-bis Cys(Acm) | protected Cys amino | |
| | acid substitutions | |
| Des 10-17 h-PYY (3- | Deletion of amino | y |
| 36)-NH₂ 5,26-bis | acids 8-15 of SEQ | |
| Cys(Acm) | ID NO 2 and cross | |
| | linkable Cys(Acm) | |
| | substitution | |
| | of SEQ ID NO 2 amino | |
| | acids 3 and 24 | |
| Des 12-16 h-PYY(3- | Deletion of amino | |
| 36)-NH₂ 5,26-bis | acids 10-14 of SEQ | |
| Cys(Acm) | ID NO 2 and cross | |
| | linkable Cys(Acm) | |
| | substitution | |
| | of SEQ ID NO 2 amino | |
| | acids 3 and 24 | |
| h-PYY-(3-36)-NH₂ | Cross linked Cys | |
| (C5-C26) | amino acid | |
| | substitutions | |
| h-PYY(3-36)-NH₂ | Cross linked Cys | |
| (C7-C24) | amino acid | |
| | substitutions | |
| h-PYY(3-36)-NH₂ | Cross linked Cys | y |
| (C3-C28) | amino acid | |
| | substitutions | |
| Des 12-16 h-PYY(3- | Deletion of amino | |
| 36)-NH₂ (C5-C26) | acids 10-14 of SEQ | |
| | ID NO 2 and cross | |
| | linked Cys | |
| | substitution of | |
| | SEQ ID NO 2 amino | |
| | acids 3 and 24 | |
| DimerDes 10-17 h- | multimer | |
| PYY (3-36)-NH₂ | | |
| (MonoACM C5, C26) | | |

Preparation of Peptide YY Analogues

It is preferred to synthesize the analogues of the invention by means of solid phase or liquid phase peptide synthesis. In this context, reference is given to WO 98/11125 and, amongst many others, Fields, G B et al., 2002, “Principles and practice of solid-phase peptide synthesis”. In: Synthetic Peptides (2nd Edition) and the Examples herein.

However, for some analogues of the invention it may be advantageous to exploit genetic engineering techniques—this may be the case when the peptide is sufficiently large (or produced as a fusion construct) and when the peptide only includes naturally occurring amino acids that can be translated from RNA in living organisms.

For the purpose of recombinant gene technology nucleic acid fragments encoding the peptides of the invention are important chemical products. Hence, an important part of the invention pertains to a nucleic acid fragment, which encodes a PYY analogue of the invention where the peptide is comprised by naturally occurring amino acids. The nucleic acid fragments of the invention are either DNA or RNA fragments.

The nucleic acid fragments of the invention will normally be inserted in suitable vectors to form cloning or expression vectors carrying the nucleic acid fragments of the invention; such novel vectors are also part of the invention. Details concerning the construction of these vectors of the invention will be discussed in context of transformed cells and microorganisms below. The vectors can, depending on purpose and type of application, be in the form of plasmids, phages, cosmids, mini-chromosomes, or virus, but also naked DNA which is only expressed transiently in certain cells is an important vector. Preferred cloning and expression vectors of the invention are capable of autonomous replication, thereby enabling high copy-numbers for the purposes of high-level expression or high-level replication for subsequent cloning.

The general outline of a vector of the invention comprises the following features in the 5′→3′ direction and in operable linkage: a promoter for driving expression of the nucleic acid fragment of the invention, optionally a nucleic acid sequence encoding a leader peptide enabling secretion (to the extracellular phase or, where applicable, into the periplasma) of or integration into the membrane of the polypeptide fragment, the nucleic acid fragment encoding the peptide of the invention, and optionally a nucleic acid sequence encoding a terminator. When operating with expression vectors in producer strains or cell-lines it is for the purposes of genetic stability of the transformed cell preferred that the vector when introduced into a host cell is integrated in the host cell genome.

The vectors of the invention are used to transform host cells to produce the modified peptide of the invention. Such transformed cells, which are also part of the invention, can be cultured cells or cell lines used for propagation of the nucleic acid fragments and vectors of the invention, or used for recombinant production of the peptides of the invention.

Preferred transformed cells of the invention are microorganisms such as bacteria (such as the species Escherichia [e.g. E. coli], Bacillus [e.g. Bacillus subtilis], Salmonella, or Mycobacterium [preferably non-pathogenic, e.g. M. bovis BCG]), yeasts (such as Saccharomyces cerevisiae), and protozoans. Alternatively, the transformed cells are derived from a multicellular organism, i.e. it may be fungal cell, an insect cell, a plant cell, or a mammalian cell. Also cells derived from a human being are interesting, cf. the discussion of cell lines and vectors below.

For the purposes of cloning and/or optimised expression it is preferred that the transformed cell is capable of replicating the nucleic acid fragment of the invention. Cells expressing the nucleic fragment are preferred useful embodiments of the invention; they can be used for small-scale or large-scale preparation of the peptides of the invention.

When producing the peptide of the invention by means of transformed cells, it is convenient, although far from essential, that the expression product is either exported out into the culture medium or carried on the surface of the transformed cell.

When an effective producer cell has been identified it is preferred, on the basis thereof, to establish a stable cell line which carries the vector of the invention and which expresses the nucleic acid fragment encoding the peptide. Preferably, this stable cell line secretes or carries the peptide of the invention, thereby facilitating purification thereof.

In general, plasmid vectors containing replicon and control sequences which are derived from species compatible with the host cell are used in connection with the hosts. The vector ordinarily carries a replication site, as well as marking sequences which are capable of providing phenotypic selection in transformed cells. For example, E. coli is typically transformed using pBR322 (but numerous other useful plasmids exist), a plasmid derived from an E. coli species (see, e.g., Bolivar et al., 1977). The pBR322 plasmid contains genes for ampicillin and tetracycline resistance and thus provides easy means for identifying transformed cells. The pBR plasmid, or other microbial plasmid or phage must also contain, or be modified to contain, promoters which can be used by the prokaryotic microorganism for expression.

Those promoters most commonly used in prokaryotic recombinant DNA construction include the β-lactamase (penicillinase) and lactose promoter systems (Chang et al., 1978; Itakura et al., 1977; Goeddel et al., 1979) and a tryptophan (trp) promoter system (Goeddel et al., 1979; EP-A-0 036 776). While these are the most commonly used, other microbial promoters have been discovered and utilized, and details concerning their nucleotide sequences have been published, enabling a skilled worker to ligate them functionally with plasmid vectors (Siebwenlist et al., 1980).

In addition to prokaryotes, eukaryotic microbes, such as yeast cultures may also be used, and also here the promoter should be capable of driving expression. Saccharomyces cerevisiae, or common baker's yeast is the most commonly used among eukaryotic microorganisms, although a number of other strains are commonly available. For expression in Saccharomyces, the plasmid YRp7, for example, is commonly used (Stinchcomb et al., 1979; Kingsman et al., 1979; Tschemper et al., 1980). This plasmid already contains the trp1 gene which provides a selection marker for a mutant strain of yeast lacking the ability to grow in tryptophan for example ATCC No. 44076 or PEP4-1 (Jones, 1977). The presence of the trp1 lesion as a characteristic of the yeast host cell genome then provides an effective environment for detecting transformation by growth in the absence of tryptophan.

Suitable promoting sequences in yeast vectors include the promoters for 3-phosphoglycerate kinase (Hitzman et al., 1980) or other glycolytic enzymes (Hess et al., 1968; Holland et al., 1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also ligated into the expression vector 3′ of the sequence desired to be expressed to provide polyadenylation of the mRNA and termination.

Other promoters, which have the additional advantage of transcription controlled by growth conditions are the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Any plasmid vector containing a yeast-compatible promoter, origin of replication and termination sequences is suitable.

In addition to microorganisms, cultures of cells derived from multicellular organisms may also be used as hosts. In principle, any such cell culture is workable, whether from vertebrate or invertebrate culture. However, interest has been greatest in vertebrate cells, and propagation of vertebrate in culture (tissue culture) has become a routine procedure in recent years (Tissue Culture, 1973). Examples of such useful host cell lines are VERO and HeLa cells, Chinese hamster ovary (CHO) cell lines, and W138, BHK, COS-7 293, Spodoptera frugiperda (SF) cells (commercially available as complete expression systems from i.a. Protein Sciences, 1000 Research Parkway, Meriden, Conn. 06450, U.S.A. and from Invitrogen), the D. melanogaster cell line S₂ available from Invitrogen, PO Box 2312, 9704 CH Groningen, The Netherlands, and MDCK cell lines.

Expression vectors for such cells ordinarily include (if necessary) an origin of replication, a promoter located in front of the gene to be expressed, along with any necessary ribosome binding sites, RNA splice sites, polyadenylation site, and transcriptional terminator sequences.

For use in mammalian cells, the control functions on the expression vectors are often provided by viral material. For example, commonly used promoters are derived from polyoma, Adenovirus 2, and most frequently Simian Virus 40 (SV40). The early and late promoters of SV40 virus are particularly useful because both are obtained easily from the virus as a fragment which also contains the SV40 viral origin of replication (Fiers et al., 1978). Smaller or larger SV40 fragments may also be used, provided there is included the approximately 250 bp sequence extending from the HindIII site toward the BgII site located in the viral origin of replication. Further, it is also possible, and often desirable, to utilize promoter or control sequences normally associated with the desired gene sequence, provided such control sequences are compatible with the host cell systems.

An origin of replication may be provided either by construction of the vector to include an exogenous origin, such as may be derived from SV40 or other viral (e.g., Polyoma, Adeno, VSV, BPV) or may be provided by the host cell chromosomal replication mechanism. If the vector is integrated into the host cell chromosome, the latter is often sufficient.

In order to obtain satisfactory yields in a recombinant production process, it may be advantageous to prepare the analogues as fusion proteins, either by fusing the peptide to a fusion partner that can serve as an affinity tag (for ease of purification) and/or by having multiple repeats of the peptide. These methods require presence of a suitable cleavage site for a peptidase, but the skilled person will know how to tailor the underlying genetic constructs.

After recombinant preparation, the peptides of the invention can be purified by methods generally known in the art, including multi-step chromatography (ion-exchange, size-exclusion, and affinity chromatographic techniques).

Alternatively, peptides comprised of naturally occurring amino acids can be prepared in vitro in cell free systems. This is especially expedient in cases where the peptides could be toxic for putative host cells. Thus, the present invention also contemplates use of cell-free in vitro translation/expression in order to prepare the peptides of the invention. In this context, reference is made to commercially available in vitro translation kits, materials, and technical documentation from e.g. Ambion Inc., 2130 Woodward, Austin, Tex. 78744-1832, USA.

Finally, the available methods can of course be combined so as to prepare e.g. semi-synthetic analogues. In such a setup, peptide fragments are prepared using at least 2 separate steps or methods, followed by ligation of the fragments to obtain the final peptide product.

To summarize, according to the present invention there is provided a method for the preparation of the peptide of the invention, which comprises
a) synthesizing the peptide by means of solid phase or liquid phase peptide synthesis and recovering the synthetic peptide thus obtained; or
b) when the peptide is constituted by naturally occurring amino acids, expressing a nucleic acid construct that encodes the peptide in a host cell and recovering the expression product from the host cell culture; or
c) when the peptide is constituted by naturally occurring amino acids, effecting cell-free in vitro expression of a nucleic acid construct that encodes the peptide and recovering the expression product; or
d) combining the methods of a, b, and c to obtain fragments of the peptide, subsequently ligating the fragments to obtain the peptide, and recovering the peptide.
   Formulation of Peptide YY Analogues
   Route of Administration

The peptides of the present invention may serve as medicaments in their pure form or as pharmaceutical compositions and they may be administered via any of the usual and acceptable methods known in the art, either singly or in combination. Such compositions may be formulated to oral administration (including buccal cavity or sublingually) or by parenteral administration (including intravenous (i.v.), subcutaneous (s.c.), intramuscular (i.m.), intraperitoneal (i.p.)) administration. Other administration routes include epidural, rectal, intranasal or dermal administration or by pulmonary inhalation.

Types of Formulations

The present invention contemplates a pharmaceutical composition comprising, as an active principle, a peptide of the invention in admixture with a pharmaceutically acceptable carrier, diluent, vehicle or excipient. Typically, such a pharmaceutical composition will be a dose form selected from the group consisting of an oral dosage form, a buccal dosage form, a sublingual dosage form, an anal dosage form, and a parenteral dosage form such as an intraveneous, an intraarterial, an intraperitoneal, a subdermal, an intradermal or an intracranial dosage form. Especially preferred formulations provide sustained release of the peptide of the invention.

The compositions may preferably be formulated to subcutaneous or oral administration, and such compositions may be prepared in a manner well known to the field. The compositions are preferably in the form of solid or liquid formulations and methods for their preparation are generally described in “Remington's Pharmaceutical Sciences”, 17th Ed., Alfonso R. Gennaro (Ed.), Mark Publishing Company, Easton, Pa., U.S.A., 1985. Solid formulations are particularly suitable for oral administration, while solutions are most useful for injection or infusion (i.v., s.c., i.m., or i.p.) or intranasal administration.

Such compositions will contain an effective amount of the one or more active peptides of this invention together with a suitable carrier in order to provide the dosage in a form compatible with the route of administration selected. The compositions comprising at least one of the peptides of this invention together with a physiologically acceptable carrier in the form of a vehicle, a diluent, a buffering agent, a tonicity adjusting agent, a preservative and stabilizers. The excipients constituting the carrier must be compatible with the active pharmaceutical ingredient(s) and preferably capable of stabilizing the peptides without being deleterious to the subject being treated.

Solid compositions may appear in conventional form such as tablets, pills, capsules, suppositories, powders or enterically coated peptides. Liquid compositions may be in the form of solutions, suspensions, dispersions, emulsions, elixirs, as well as sustained release formulations, and the like. Topical compositions may be in the form of plasters or pastes and inhalation compositions may be contained in spray delivery systems.

Depot (Sustained Release) Formulations

In a preferred embodiment of the invention depot formulations that include at least one of the present peptides are envisioned. A form of repository or depot formulation may be used so that therapeutically effective amounts of the preparation are delivered into the bloodstream over many hours or days following transdermal injection or deposition. Formulations suitable for sustained release formulations include biodegradable polymers and may consist of appropriate biodegradable polymers, such as L-lactic acid, D-lactic acid, DL-lactic acid, glycolide, glycolic acid, and any isomers thereof. Similarly, the carrier or diluent may include any sustained release material known in the art, such as glyceryl monostearate or glyceryl distearate, alone or mixed with a wax.

Other depot formulations may include, but are not limited to, formulations that include at least one of the present peptides disclosed herein combined with liposomes, microspheres, emulsions or micelles and liquid stabilizers.

Aqueous Formulation

Aqueous formulations of the peptides of this invention may be prepared for parenteral administration by injection or infusion (i.v., s.c., i.m. or i.p.). Since the peptides of the invention are amphoteric, they may be utilized as free acids or bases, or as salts. The salts must, of course, be pharmaceutically acceptable, and these will include alkali and metal salts of acidic peptides, e,g., potassium, sodium or magnesium salts. The salts of basic peptides will include salts of halides and inorganic and organic acids, e.g. chloride, phosphate or acetate. Salts of the peptides are readily prepared by procedures well known to those skilled in the art.

The peptides of this invention may be provided as liquid or semi-liquid compositions for parenteral administration (e.g. injection, infusion or deposition of slow release depot formulations). The peptides may be suspended or dissolved in an aqueous carrier, for example, in a suitably buffered solution at a pH of about 3.0 to about 8.0, preferably at a pH of about 3.5 to about 7.4, 3.5 to 6.0, or 3.5 to about 5.0. Useful buffers include sodium citrate/citric acid, sodium phosphate/phosphoric acid, sodium acetate/acetic acid, or combinations thereof.

Such aqueous solutions may be rendered isotonic by adjusting the osmotic pressure with a buffering agent, by the inclusion of saline, aqueous dextrose, glycols or by the use of sugars such as lactose, glucose or mannitol and the like.

The compositions may be other pharmaceutically acceptable excipients such as preservatives, stabilizing agents, and wetting or emulsifying agents as described in “Handbook of Pharmaceutical Excipients”, 3^{rd} Ed., Arthur H. Kibbe (Ed.), Pharmaceutical Press, London, UK (2000). The preservatives may include sodium benzoate, sodium sorbic acid, phenol or cresols and parabens. Stabilizing agents may include carboxymethyl-cellulose, cyclodextrins or detergents.

The preparation may be produced immediately before use from active drug substance and sterile carrier solution. Alternatively, the compositions may be filled into sealed glass vials or ampoules, and if necessary purged with an inert gas, under aseptic conditions and stored until needed. This allows for continued multi-dose therapy but also demands the highest degree of stability of the compound.

Oleaginous Formulations

Oleaginous formulations of the peptides of this invention may be prepared for parenteral administration by injection (s.c., i.m. or i.p.) or topically. The carrier can be selected from the various oils including those of petroleum, animal, vegetable or synthetic origin, e.g., peanut oil, soybean oil, mineral oil, sesame oil, and the like. The compositions may be in the form of solutions or suspensions. Solutions of the peptides may be prepared with the use of detergents and emulsifiers and suspensions may be prepared using powder or crystalline salts. The compositions may be stabilized with preservatives (e.g. butylated hydroxianisole or butylated hydroxytoluene).

Nasal Administration

For nasal administration by pulmonary inhalation, the formulation may contain one or more peptides of the present invention, dissolved or suspended in a liquid carrier, in particular, an aqueous carrier, for aerosol application. The carrier may contain auxiliary additives such as solubilizing agents, e.g., propylene glycol, surfactants such as polyoxyethylene, higher alcohol ethers, and absorption enhancers such as lecithin or cyclodextrin and preservatives such as sorbic acid, cresols or parabens.

Topical Formulations

Topical administration for local application and action of the peptides of this invention may be in the form of pastes prepared by dispersing the active compound in a pharmaceutically acceptable oil such as peanut oil, sesame oil, corn oil or the like. Alternatively, the peptides may be incorporated into patches for dermal administration. Patches may be prepared in a form for iontophoretic application.

Suppositories

Suppositories for transmucosal administration may be in the form of pellets containing an effective amount of a compound of the present invention can be prepared by admixing a compound of the present invention with a diluent such as carbowax, carnuba wax, and the like, and a lubricant, such as magnesium or calcium stearate.

Oral Formulations

Solid compositions are preferred for oral administration in the form of tablets, pills, capsules, powders, and the like. Tablets may contain stabilizing buffering agents (e.g. sodium citrate, calcium carbonate and calcium phosphate), disintegrants (e.g. potato or tapioca starch, and complex silicates) binding agents (e.g. polyvinylpyrrolidone, lactose, mannitol, sucrose, gelatin, agar, pectin and acacia) and lubricating agents (e.g. magnesium stearate, stearic acid or sodium lauryl sulfate) as well as other fillers (e.g. cellulose or polyethylene glycols). Liquid formulations for oral administration may be combined with various sweetening agents, flavoring agents, coloring agents, in addition to diluents such as water, ethanol, propylene glycol, glycerin.

Doses

The doses the peptides and compositions of the present invention required for the desired therapeutical effects will depend upon on the potency of the compound, the particular composition used and the route of administration selected. The peptides will typically be administrated in the range of about 0.001 to 10 g per patient per day, preferably from about 1 to about 1000 mg per patient per day, more preferably from about 10 to about 100 mg per patient per day, about 50 mg per patient per day. Dosages for certain routes, for example oral and other non-parenteral administration routes, should be increased to account for any decreased bioavailability, for example, by about 5-100 fold.

Dosing Regimen

The most suitable dosing regimen may best be determined by a medical practitioner for each patient individually. The optimal dosing regimen with the peptides and pharmaceutical compositions of this invention depends on factors such as the particular disease or disorder being treated, the desired effect, and the age, weight or body mass index, and general physical conditions of the patient. The administration may be conducted in a single unit dosage form to alleviate acute symptoms or as a continuous therapy in the form of multiple doses over time. Alternatively, continuous infusion systems or slow release depot formulations may be employed. Two or more peptides or pharmaceutical compositions of this invention may be co-administered simultaneously or sequentially in any order. In addition, the peptides and compositions may be administered in a similar manner for prophylactic purposes. The best dosing regimen will ultimately be decided by the attending physician for each patient individually.

The following non-limiting examples are presented merely in order to illustrate the invention. The skilled person in the area will understand that there are numerous equivalents and variations not exemplified but still forming part of the present invention.

Use of the PYY Analogues in Disease Treatment

The present invention contemplates in one embodiment a method for reducing body weight in a subject, the method comprising administering, to the subject, an effective amount of the peptide or pharmaceutical composition of the invention.

In a further embodiment the present invention relates to a method for enhancing body weight in a subject, the method comprising administering, to the subject, an effective amount of the peptide or pharmaceutical composition of the invention.

As will be appreciated from the above, administration of the inventive analogues is expected to in one embodiment providing effective means for reducing excess body fat in individuals in need thereof, and in another embodiment providing effective means for increasing body fat in individuals in need thereof. It is contemplated that the presently suggested therapeutic treatment of humans should be accompanied by a controlled diet in order to ensure that the person undergoing treatment ingests necessary nutrients. At the same time the rate of weight loss or weight gain should be carefully monitored in order to avoid too drastic reductions or increases in body weight over time and it should be ensured that the treated subject exerts a physical behaviour that aims at preserving muscle mass etc.

Overweight and obese individuals (BMI of 25 and above) are at increased risk for physical ailments such as: High blood pressure, hypertension; High blood cholesterol, dyslipidemia; Type 2 (non-insulin dependent) Diabetes; Insulin resistance, glucose intolerance; Hyperinsulinemia; Coronary heart disease; Angina pectoris; Congestive heart failure; Stroke; Gallstones; Cholescystitis and cholelithiasis; Gout; Osteoarthritis; Obstructive sleep apnoea and respiratory problems; Musculo-skeletal diseases; Some types of cancer (such as endometrial, breast, prostate, and colon); Complications of pregnancy; Poor female reproductive health (such as menstrual irregularities, infertility, irregular ovulation); Bladder control problems (such as stress incontinence); Uric acid nephrolithiasis; Psychological disorders (such as depression, eating disorders, distorted body image, and low self esteem). Obesity is also a risk-factor for the group of metabolic derangements collectively named the metabolic syndrome or “Syndrome X”. The health consequences of obesity range from increased risk of premature death to serious chronic conditions that reduce the overall quality of life. Furthermore, severe obesity is associated with a 12 fold increase in mortality in 25-35 year olds when compared to lean individuals. Negative attitudes towards the obese can lead to discrimination in many areas of their life including health care and employment.

Since the present invention in one aspect provides means for reducing body fat deposits, any one of the above-listed syndromes, diseases and conditions are targets for the aspect of the invention that relates to therapy and prophylaxis and the inventive peptides are useful against any disease or condition characterized by excess body fat deposition.

In a further aspect as mentioned above, administration of the present analogues is expected to provide effective means for enhancing/increasing body fat in individuals in need thereof. Thus, the present invention also concerns peptides used to treat or ameliorate conditions characterised by reduced body fat deposition and for the preparation of a pharmaceutical composition for the treatment or amelioration of conditions characterized by reduced body fat deposition. By reduced body fat deposition is meant a very low body fat deposition as seen in individuals suffering from for example eating disorders, such as anorexia and bulimia. Low body fat may also be observed in individuals suffering from medical conditions, wherein loss of appetite and thereby loss of body fat is an either direct or indirect effect of said medical condition. One such condition is cancer related cachexia. The present peptides may be used to induce appetite in individuals in need thereof.

Administration of the peptide or composition of the invention is preferably via a route selected from the group consisting of the parenteral route such as the intradermal, the subdermal, the intraarterial, the intravenous, and the intramuscular route; the peritoneal route; the oral route; the buccal route; the sublinqual route; the epidural route; the spinal route; the anal route; and the intracranial route.

The effective amount will be determined by the skilled person taking into account such factors as potency of the drug, age and constitution of the patient, body weight, pharmacokinetic profile of the drug, and in general the drug will be prescribed for each patient or group of patients. However, the effective amount of the peptide is preferably at least about 10 μg/kg body weight/day, such as at least 100 μg/kg body weight/day, at least 300 μg/body weight/day, and at least 1000 μg/kg body weight/day. On the other hand, the effective amount of the peptide or dimer is preferably at most about 100 mg/kg body weight/day, such as at most 50 mg/kg body weight/day and at most 10 mg/kg body weight/day. It is expected that the effective amount of the peptide will be about 100 μg/kg body weight/day, about 300 μg/kg body weight/day or about 1000 μg/kg body weight/day.

### EXAMPLE 1

### Peptide Synthesis

A preferred general procedure is described below. However, more detailed descriptions of solid phase peptide synthesis methods are found in WO 98/11125 hereby incorporated in its entirety.

General Peptide Synthesis

Abbreviations

Acm Acetamidomethyl
Boc tertbutyloxycarbonyl
Dbf 4-(2-Aminoethyl)6-dibenzofuranpropionic acid
DIC Diisopropylcarbodidimide
DMF N,N-Dimethylformamide
EDT Ethanedithiol
Fmoc Fluorenyl methyloxycarbonyl
HOBt 1-Hydroxybenzotriazole
ivDde (4,4-dimethyl-2,6-dioxocyclohex-1-ylidene)-3-methylbutyl
Rₜ Retention time
TFA Trifluoroacetic acid

### Chemicals

Protected amino acids, and HOBtxH₂O were purchased from Advanced Chemtech (Louisville Ky. USA). FMOC-Dbf-OH was from Neosystem (Strassbourg, France), Fmoc-Lys(ivDde)-OH was from Bachem (Bubendorf Switzerland); Tentagel-S-Ram-Fmoc resin was from Rapp Polymere (Tübingen, Germany). TFA was from Halocarbon (River Edge N.J. USA). DIC, and hydrazine hydrate was from Fluka (Buchs Switzerland). EDT was from Aldrich (St. Louis, Mo. USA). Acetonitrile was from SDS (Toulouse France). Other solvents were of technical quality but distilled.

### Analytical HPLC:

Instrument: Agilent Technologies 1100 liquid chromatograph consisting of an on-line degasser, a binary gradient pump, a temperature controllable autosampler, a column oven and a diode array UV detector. The chromatograph was controlled by chemstation software Rev. A 8.03 (Agilent Technologies, Waldbron, Germany).
Column: Kromasil RP C8; K 100-10-CS 250×4.6 mm.
Detection 215 and 254 nm. Integration at 215 nm
Temperature: 40° C.
Flow: 1.0 ml/min
Buffers: A: 0.10% TFA in water; B: 9.90% water, 0.10% TFA 90.0% acetonitrile
Gradient: Start 100% A, 0-1.5 min 100% A 1.5-25 min 0-50% B

### Preparative HPLC:

Instrument: Vision Workstation from PerSeptive Biosystems Inc.
Column P5 Kromasil RP C8; K 100-10-C8 250×50.8 mm.
Detection 215 and 280 nm.
Temperature ambient approx. 20° C.
Flow 35 ml/min
Buffers: A: 0.10% TFA in water; B: 9.90% water, 0.10% TFA 90.0% acetonitrile
Fraction size: 9 ml
Gradient: Start 100% A, 0-3 min 0-10% B, 3-53 min 10-60% B

### Mass Spectroscopy

The peptides were dissolved in methanol, water and formic acid (50:50:0.1 v/v/v) to give concentrations between 1 and 10 μg/ml. The peptide solutions were analysed in positive polarity mode by ESI-TOF-MS using a LCT mass spectrometer (Micromass, Manchester, UK) accuracy of +/−0.1 m/z.

### General Synthetic Procedure

In all syntheses, dry TentaGel-S-Ram-Fmoc resin (1 g, 0.22-0.31 mmol/g) was placed in a polyethylene vessel equipped with a polypropylene filter for filtration. The resin was swelled in DMF (15 ml), and the FMoc group removed (see below). The resin was drained and washed with DMF until no yellow colour could be detected after addition of Dhbt-OH to the drained DMF. The amino acids according to the sequence were coupled as preformed Fmoc-protected HOBt esters (3 eq.) (see below). The coupling was 2 h or over night as convenient. The resin was drained and washed with DMF (5×15 ml, 5 min each) in order to remove excess reagent. All acylations were checked by the Kaiser test. In case of a positive test double coupling was performed. Otherwise deprotection (see below) was performed and the next protected amino acid coupled to the peptidyl resin. After completed synthesis the peptidyl resin was washed with DMF (3×15 ml, 5 min each), DCM (3×15 ml, 1 min each) and finally diethyl ether (3×15 ml, 1 min each) and dried in vacuo. The peptide was then cleaved from the resin as described below.

After purification using preparative HPLC as described above, the fractions containing the purified peptide were collected and lyophilised to yield the peptide as its trifluoroacetate. All products made were white powders. The identity of the peptide was confirmed by ES-MS and the purity by HPLC. This procedure was used for the synthesis of all peptides exemplified further below.

### Deprotection of the N-α-Amino Protecting Group (Fmoc)

Deprotection of the Fmoc group was performed by treatment with 20% piperidine in DMF (1×5 and 1×10 min.), followed by wash with DMF (5-10×15 ml) until no yellow colour could be detected after addition of Dhbt-OH to the drained DMF.

### Coupling of HOBt-Esters

3 eq. N-α-amino protected amino acid was dissolved in DMF together with 3 eq. HOBt and 3 eq. DIC and then added to the resin.

### Cleavage of Peptide from Resin With Acid

Peptides were cleaved from the resins by treatment with 95% TFA and 5% EDT v/v at ambient temperature for 2 hours 20 ml/g resin). The filtered resins were further extracted with TFA (3×10 ml) and the combined TFA fractions were evaporated under reduced pressure to approximately 4 ml. Ether (60-70 ml) was added to precipitate the crude peptide as its trifluoroacetate. It was analysed by HPLC and identified by electro spray ionisation mass spectrometry (ESMS).

### Synthesis of Individual Peptides

SEQ ID 2, SEQ ID 3, SEQ ID 4, SEQ ID 5, SEQ ID 10, SEQ ID 11, SEQ ID 16, SEQ ID 17, SEQ ID 18, and SEQ ID 19 were all assembled according to the “general synthetic procedure” described above.

SEQ ID 6, SEQ ID 7, SEQ ID 8, SEQ ID 9, SEQ ID 12, SEQ ID 13, SEQ ID 14, and SEQ ID 15 were all assembled according to the “general synthetic procedure” described above using cysteines protected on sulphur with Acm. The purified Acm protected peptide (20 mg) and silver trifluoroacetate (20 mg) were dissolved in TFA (0.50 ml) in a 15 ml centrifuge tube and anisole (5 micro litre) was added. The solution was left over night and ether (5 ml) was added to precipitate the peptide with free SH groups as its silver salt. The precipitate was washed once with ether (2 ml) and dissolved in water (5 ml). 2 M HCl (5 ml) was added. The solution became turbid due to precipitation of silver chloride. DMSO (3 ml) was added to oxidise the sulfhydryl groups to disulfide bonds. The reaction was followed by HPLC and in all cases complete within 18 h. The silver chloride was spun down by centrifugation and the clear supernatant loaded directly on the preparative HPLC and purified.

SEQ ID 20, SEQ ID 21, and SEQ ID 22 were assembled according to the “general synthetic procedure” described above using Fmoc-Lys(ivDde)-OH at position 8 from the C terminus in the backbone. The amino acid terminating the backbone was coupled as its Boc protected derivative. Then the ivDde group was removed from the epsilon amino group by treatment of the resin with 2% hydrazine hydrate in DMF for 3×3 min. After washing of the resin synthesis was continued as described above only that it was now the epsilon amino group on lysine onto which the growing peptide chain extended.

### Result From the Syntheses

All masses were in accordance with theory ±0.5 Dalton

| SEQ ID | Yield# in % | Rt § in min | MW mono |
|---|---|---|---|
| 2 | 1.3 | 22.2 | 4047.07 |
| 3 | 2.1 | 20.1 | 4497.44 |
| 4 | 1.1 | 20.7 | 4815.64 |
| 6 | 14.5 | 18.8 | 4038.92 |
| 10 | 3 | 23.7 | 4126.12 |
| 11 | 5 | 22.5 | 4055.11 |
| 12 | 10.2 | 18.4 | 3525.71 |
| 13 | 10.7 | 17.5 | 3168.58 |
| 16 | 3 | 22.3 | 3039.58 |
| 17 | 7.8 | 20.6 | 3808.15 |
| 18 | 36 | 18.5 | 1888.05 |
| 19 | 17 | 15.5 | 2656.62 |
| 20 | 5.5 | 21.8 | 3591.94 |
| 21 | 3.5 | 18.4 | 2440.42 |
| 22 | 9.7 | 16.8 | 3141.79 |

#purified yield relative to Resin load
§ Gradient as descibed in HPLC analytical.

### EXAMPLE 2

### Pre-Screening of PYY Analogues

The PYY analogues of the present invention are pre-screened in the following in vitro assays set forth in this and the following Example.

Y₁ and Y₂ Receptor Binding Assay

Cell membranes (5-10 μgₚᵣₒₜ) derived from SK-N-MC, SK-N-BE(2), SMS-KAN or brain cortex from adult rats are incubated with 0.2 nM [¹²⁵I] {Leu31,Pro34}PYY (Y₁-ligand) or 0.2 nM [¹²⁵I]PYY₃₋₃₆ (Y₂-ligand) in the absence or presence of increasing concentrations of test peptides in 200 μl binding buffer (50 mM HEPES, 2.5 mM CaCl₂, 1 mM MgCl₂, & 0.1% BSA, pH 7.2). Non specific binding are estimated at 1 μM {Leu3 1,Pro34}PYY (Y₁) respectively PYY₃₋₃₆ (Y₂).

The assay mixtures are incubated for 90 min at either 30° C. (Y₁-binding) or room temperature (Y₂-binding) followed by rapid filtration on Unifilters (GF/C), pre-soaked in 0.5% polyethylenimin for at least 30 min before use. The filters are washed twice with 150 μl ice-cold D-PBS, dried for 60 min at 60° C., scintilation cocktail added and counted in a TopCount scintilation counter. IC₅₀-values are estimated by computer aided curve fitting.

Y₁ and Y₂ Receptor Efficacy Assay

SK-N-.MC, SK-N-BE(2) or SMS-KAN cells are seeded at 20,000 cells per well in 96-well microtiter plates and grow for 3 days in culture to confluency. On the day of analysis growth medium is removed and the cells washed once with 200 μl Tyrode buffer. Cells are incubated in 100 μl Tyrode buffer containing increasing concentrations of test peptides, 100 μM IBMX, 6 mM glucose and either 1 μM (SK-N-MC) or 10 μM (SK-N-BE(2) or SMS-KAN) forskolin for 30 min at 37° C. The reaction is stopped by addition of 25 μl 0.5 M HCl and incubation on ice for 60 min. The cAMP content is estimated using the FlashPlate® cAMP kit from PerkinElmer. EC₅₀ and relative efficacy are estimated by computer aided curve fitting.

### EXAMPLE 3

### Y₅ Receptor Binding Assay

Screening of the present PYY analogues in a receptor Y₅ binding assay can be performed as described by Norman M. H. et al. in J. Med. Chem, 2000, 43, 4288-4312, which is hereby incorporated by reference herein.

### EXAMPLE 4

### The Effect of the Y2 Receptor Preferring PYY Ligands on Food Intake (1, 2, 3, 4 and 24 Hours Following Injection) in Overnight Fasted Male C57BL/6J Mice

The acute effects of the present PYY analogues on food intake in overnight fasted C57BL/6J mice fed high or low-fat diets are examined.

Experimental Protocol

Animals

Thirty male C57BL/6J mice (Charles River) 4-5 weeks old at the time of arrival are used. The mice are 5 per cage for 5 weeks then transferred to individual cages. Fifteen mice are fed ad libitum with a low fat diet (Low fat D12489B, Research Diets Inc, New Brunswick, USA) and 15 mice are fed ad libitum with a high fat diet (High fat D12266B, Research Diets Inc, New Brunswick, USA) housed singly before experiments are performed. Free access to food and water unless otherwise stated in a temperature controlled room (20-22° C.); L/D cycle of 12/12 (lights on at 0400).

Peptides and Vehicle

All test-peptides are dissolved in vehicle (0.9% NaCl, physiological saline, pH=7.4)
Test peptides: PYY analogues of the invention
Groups (n=7-8)
Group 1: Low-fat, Vehicle
Group 2: Low-fat, PYY analogueue 100 μg/kg
Group 3: High-fat, Vehicle
Group 4: High-fat, PYY analogueue 100 μg/kg

The compound is dissolved to reach a final concentration of 50 μg/ml.

Experimental Set-Up

For the first 5 weeks mice are kept 5 per cage (tail marked to identify individual mice). Mice are weighed once weekly. On week 6, mice are transferred to individual cages (still with free access to food and water) and kept for 7 days. For 3 days prior to the first experimental day the animals are injected at 9:00 am with 0.1 ml saline. On the day prior to the experiment the animals are randomised (according to body-weight) into the four treatment groups. Food is removed and weighed (each individuals mouse food is kept in a labelled container) at 15:00 pm and the mice are fasted for the subsequent 19 hours (water is available ad libitum throughout the experiment). In the morning the following day at 9:00 am the mice are injected with the test substances and given their pre-weighed food back when they are returned to the cage. Food is weighed 1, 2, 3, 4 and 24 hours after the injection (that is at 10:00 am, 11:00 am, 12:00 am, 1:00 pm and 9:00 am the next day).

### EXAMPLE 5

### The Effects of 26 Days of Administration of PYY3-36 and Analogues on Body-Weight in Diet-Induced Obese Male C57BL/6J Mice

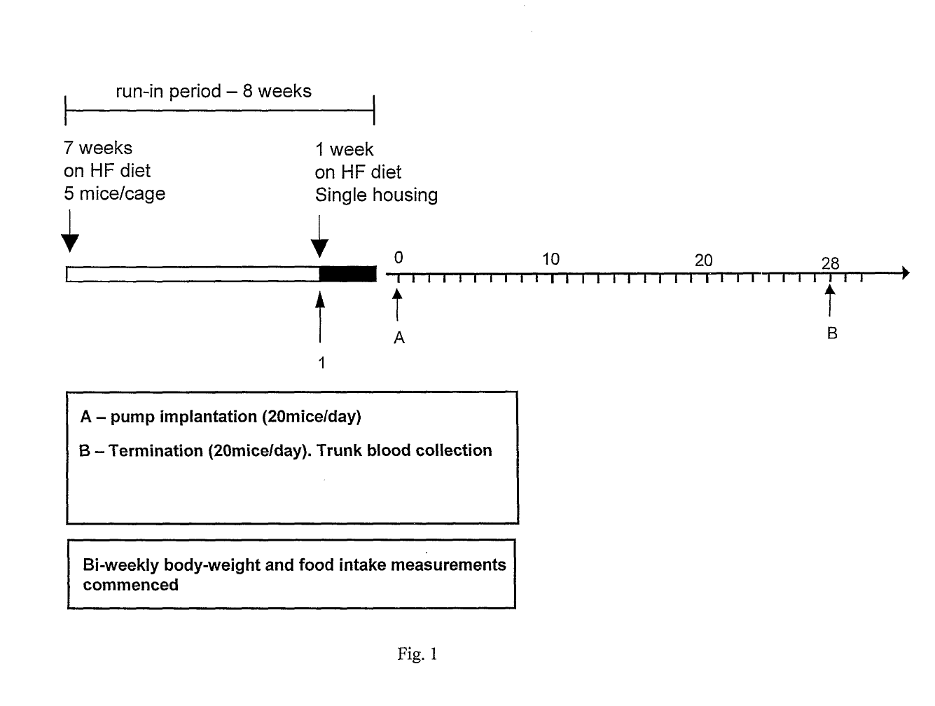

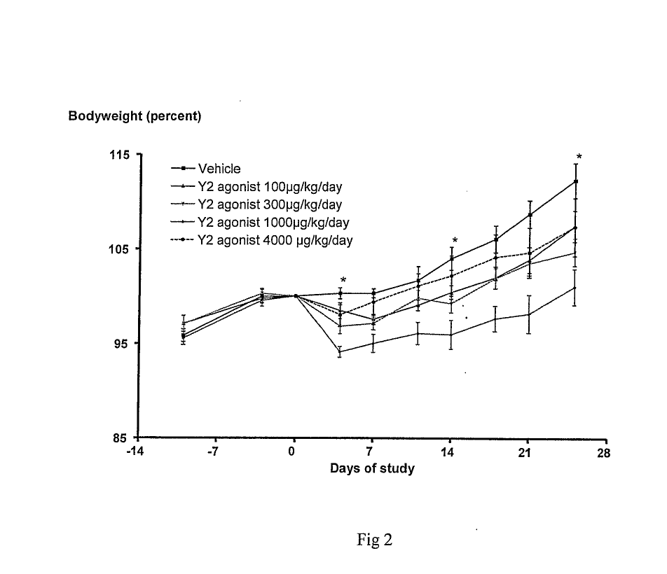

The effect of subchronic (28 days) continuous (subcutaneous) administration of an Y2 receptor preferring PYY analogue on body-weight in high-fat fed C57BL/6J mice is examined. The experimental procedure is set forth in FIG. 1 and results are shown in FIG. 2.

Animals

Forty male C57BL/6J mice (Charles River) 4-5 weeks old at the time of arrival are used. The mice are housed 5 per cage for 7 weeks then transferred to individual cages for the remainder of the experiment. For the entire experiment the mice have free access to high fat diet (4.41 kcal/g—Energy %: Carbohydrate 51.4 kcal %, Fat 31.8 kcal %, Protein 16.8 kcal %; diet #12266B; Research Diets, New Jersey, USA).

Peptides and Vehicle

All test-peptides are dissolved in vehicle (0.9% NaCl,=physiological saline, pH=7.4)
Test peptides: PYY analogues of the present invention
Groups (n=10)
Group 1: Vehicle
Group 2: PYY 3-36 100 μg/kg/day
Group 3: PYY 3-36 300 μg/kg/day
Group 4: PYY 3-36 1000 μg/kg/day

Peptides are administered via Alzet osmotic minipumps (model 2004; 200 μl; 0.25 μl/h, 28 days of delivery). The final concentration is calculated according to the following formulas (is calculated on the basis of the average body-weight “group average BW” of each group)
Group 2: 100 μg/kg/day
Concentration (μg/ml)=(100 μg/kg/day*(group 2 average BW)kg*28 days)/0.2 ml
Group 3: 300 μg/kg/day
Concentration (μg/ml)=(300 μg/kg/day*(group 3 average BW)kg*28 days)/0.2 ml
Group 4: 1000 μg/kg/day
Concentration (μg/ml)=(1000 μg/kg/day*(group 4 average BW)kg*28 days)/0.2 ml

Pumps are filled on day-1 and “primed” overnight according to the manufactures recommendation (pump filled and kept in 0.9% saline at 37° C. overnight, approximately 19 hours).

Experimental Set-Up

For the first 7 weeks mice are kept 5 per cage. Beginning week 8 mice are transferred to individual cages and body-weight and food intake is monitored bi-weekly. On Experimental day -1 animals are weighed and randomized according to body-weight into the 4 treatment groups. On day 0 animals are anaesthatized using gas anaesthesy (Isofluran) and Alzet osmotic pumps (model 2004) implanted subcutaneously in the lower back. Following the operation, mice are allowed to recover, then transferred back to their cages. For the following 26 days, food intake and body-weight is monitored bi-weekly. On the morning of day 27, mice are killed by decapitation and trunk blood is collected in EDTA plasma vials. Plasma is stored at −20 degrees Celsius until further analysis (Triglycerides, Cholesterol and Glucose).

The results show dose related results consistent with activation of the Y2 receptor progressively increasing with dose over the range 0-1000 μg/kg/day range and decreasing upon going to 4000 μg/kg/day, probably due to increased stimulation of the Y1 receptor as the size of the dose overcomes the Y2 selectivity of the medicament.

1-75. (canceled)

76. A peptide, which is a sequence variant and a functional and/or structural mimic of peptide YY, said peptide comprising at least one modification of the amino acid sequence set forth in SEQ ID NO: 2 (h-PYY 3-36), wherein said peptide
includes a modification that conformationally constrains the relative position of the N-terminal amino acid of that part of SEQ ID NO 2 present in the peptide and amino acid 34 of SEQ ID NO: 2 in the peptide; and/or
includes a branched amino acid sequence resulting in 2 free N-terminal amino acids; and/or
includes N-terminal and/or C-terminal addition of a net basic amino acid sequence;
optionally further includes deletion of amino acids 1-5 of SEQ ID NO: 2; and/or
includes deletion of any one or more of amino acid residues 8-15 of SEQ ID NO: 2 without deletion of all of amino acids 1-7 of SEQ ID NO 2; and/or
includes deletion of amino acids 6 and 7 of SEQ ID NO: 2 without deletion of all of amino acids 1-5 of SEQ ID NO 2; and/or
includes deletion of amino acids 16-19 of SEQ ID NO: 2 without deletion of all of amino acids 1-15 of SEQ ID NO 2; and/or
includes two cross linkable protected Cys amino acid substitutions; wherein said peptide further comprises at most 6 substitutions in the amino acid sequence set forth in SEQ ID NO: 2, each of which is a structure and/or functionality preserving substitution.

77. The peptide according to claim 76, wherein the modification that conformationally constrains the relative position of amino acids 1 and 34 of SEQ ID NO: 2 is selected from the group consisting of introduction of a disulfide bridge, introduction of a rigid bend involving positions corresponding to residues 9 and 10 in SEQ ID NO: 2, and introduction of at least one stabilizing amide bond between amino acid side chains.

78. A peptide of formula I
R¹-X-Y-Z-A²²-A²³-A²⁴-A²⁵-A²⁶-A²⁷-A²⁸-A²⁹-A³⁰-A³¹-A³²-A³³-A³⁴-A³⁵-A³⁶-R² (I)
wherein
A²² is Ala or a structure and/or functionality preserving substitution thereof;
A²³ is Ser or a structure and/or functionality preserving substitution thereof;
A²⁴ is Leu or a structure and/or functionality preserving substitution thereof, His or Cys;
A²⁵ is Arg or a structure and/or functionality preserving substitution thereof;
A²⁶ is Leu or a structure and/or functionality preserving substitution thereof, His or Cys;
A²⁷ is Tyr or a structure and/or functionality preserving substitution thereof;
A²⁸ is Leu or a structure and/or functionality preserving substitution thereof, or Cys;
A²⁹ is Asn or a structure and/or functionality preserving substitution thereof; or Lys, which is optionally coupled to an amino acid sequence via a peptide bond at the c-amino group;
A³⁰ is Leu or a structure and/or functionality preserving substitution thereof;
A³¹ is Val or a structure and/or functionality preserving substitution thereof, or Cys;
A³² is Thr or a structure and/or functionality preserving substitution thereof;
A³³ is Arg or a structure and/or functionality preserving substitution thereof;
A³⁴ is Gln or a structure and/or functionality preserving substitution thereof;
A³⁵ is Arg or a structure and/or functionality preserving substitution thereof; and
A³⁶ is Tyr or a structure and/or functionality preserving substitution thereof;
Z is a peptide of formula
A¹³-A¹⁴-A¹⁵-A¹⁶-A¹⁷-A¹⁸-A¹⁹-A²⁰-A²¹
which is absent or wherein,
A¹³ is Ser or a structure and/or functionality preserving substitution thereof or absent;
A¹⁴ is Pro or a structure and/or functionality preserving substitution thereof or absent;
A¹⁵ is Glu or a structure and/or functionality preserving substitution thereof or absent;
A¹⁶ is Glu or a structure and/or functionality preserving substitution thereof or absent;
A¹⁷ is Leu or a structure and/or functionality preserving substitution thereof or absent;
A¹⁸ is Asn or a structure and/or functionality preserving substitution thereof;
A¹⁹ is Arg or a structure and/or functionality preserving substitution thereof;
A²⁰ is Tyr or a structure and/or functionality preserving substitution thereof; and
A²¹ is Tyr or a structure and/or functionality preserving substitution thereof;
Y is a peptide of formula
A⁸-A⁹-A¹⁰-A-B
which is absent or wherein
A⁸ is Pro or a structure and/or functionality preserving substitution thereof;
A⁹ is Gly or a structure and/or functionality preserving substitution thereof;
A¹⁰ is Glu or a structure and/or functionality preserving substitution thereof, or absent; and
A-B designates a dipeptide A¹¹-A¹² selected from the group consisting of Gly-Gly, Pro-Gly, Gly-Pro, Sar-Sar, Sar-Hyp, Hyp-Sar, Pro-Sar, Sar-Pro, Pro-Hyp, Pro-Pro, Hyp-Pro, and Hyp-Hyp, where Pro and Hyp independently may be an L or D form, where the ring structure of Pro (III) and Hyp is optionally substituted with halogen, nitro, methyl, amino, or phenyl, Hyp represents 3-hydroxyproline or 4-hydroxyproline, Sar represents sarcosine, or on e or both of the amino acid residues of A-B is a Sar, or an N-cyclohexylglycine residue, or A and B each independently represents a group of the formula II 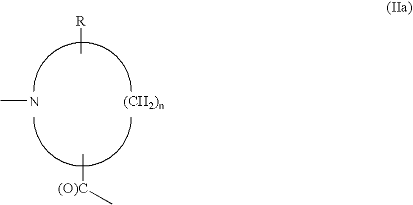
wherein n is an integer having the value 3, 4, or 5, and R represents an optional substituent, preferably selected from the group consisting of halogen, phenyl, hydroxy, NH₂, and C(1-6)alkyl optionally substituted with halogen, or A-B designates the formula IIa 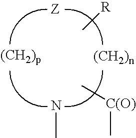
wherein n is an integer having the value 0, 1, 2, and 3, p is an integer having the value 0, 1, 2, and 3, Z represents 0 or S, and R represents an optional substituent, preferably selected from the group consisting of halogen, phenyl, hydroxy, NH₂, and C(1-6)alkyl, or A and B independently represents an amino acid residue having a saturated carbocyclic structure of 4, 5 or 6 members and where in said carbocyclic structure further comprises one or more heteroatoms,
X is a peptide of formula
A³-A⁴-A⁵-A⁶-A⁷
which is absent or wherein
A³ is Ile or a structure and/or functionality preserving substitution thereof, or Cys;
A⁴ is Lys or a structure and/or functionality preserving substitution thereof;
A⁵ is Pro or a structure and/or functionality preserving substitution thereof, or Cys;
A⁶ is Glu or a structure and/or functionality preserving substitution thereof; and
A⁷ is Ala or a structure and/or functionality preserving substitution thereof, or Cys;
R¹ is absent or an amino acid sequence; and
R² is absent or an amino acid sequence;
wherein said peptide comprises at most one disulfide bridge selected from Cys³-S—S-Cys³¹, Cys³-S—S-CyS²⁸, CyS⁵-S—S-CYS²⁶, and Cys⁷-S—S-CYS²⁴
or wherein A is absent, Asp or a structure and/or functionality preserving substitution thereof and B is absent, Ala or a structure and/or functionality preserving substitution thereof and said peptide comprises a disulfide bridge selected from Cys³-S—S-Cys³¹, Cys³-S—S-Cys²⁸, Cys⁵-S—S-Cys²⁶, and Cys⁷-S—S-Cys²⁴;
wherein the number of structure and/or functionality preserving substitutions does not exceed 6;
wherein the C-terminal amino exposes a free carboxylic acid group or an amide group; and
or a multimer and/or pharmaceutically acceptable salt thereof.

79. The peptide according to claim 76, which binds with higher affinity to receptor Y2 than to receptor Y1.

80. The peptide according to claim 76, which binds with higher affinity to receptor Y5 than to receptor Y 1.

81. The peptide according to claim 78, wherein A²⁹ is Lys.

82. The peptide according to claim 81, wherein Lys²⁹ is coupled to an amino acid sequence via a peptide bond at the E-amino group.

83. The peptide according to claim 78, wherein at most one of A²⁴, A²⁶, A²⁸, and A³¹ is Cys.

84. The peptide according to claim 78, comprising the disulfide bridge Cys³-S—S-Cys³¹, or comprising the disulfide bridge Cys³-S—S-Cys²⁸, or comprising the disulfide bridge Cys⁵-S—S-Cys26, or comprising the disulfide bridge Cys⁷-S—S-Cys24.

85. The peptide according to claim 78, wherein X has the amino acid sequence set forth in SEQ ID NO: 23 or wherein X is absent.

86. The peptide according to claim 78, wherein A and B, independently are selected from the group consisting of N- and C(O)-radicals of the following compounds:
D/L-azetidin-3-carboxylic acid,
D/L-azetidin-2-carboxylic acid,
D/L-Indolin-2-carboxylic acid,
D/L-1,3-dihydro-isoindol-1-carboxylic acid,
D/L-thiazolidin4-carboxylic acid,
D/L-pipecolinic acid,
D/L-nipecotinic acid,
isonipecotinic acid,
L/D-2-carboxymorpholin,
L/D-1,2,3,4-tetrahydroquinolin-3-carboxylic acid,
L/D-1,2,3,4-tetrahydroquinolin-3-carboxylic acid, and
4-carboxy4-phenyl-piperidin.

87. The peptide according to claim 78, wherein A-B designates 4-(2-aminoethyl)-6-dibenzofuranpropionic acid.

88. The peptide according to claim 78, wherein A-B is a dipeptide or wherein A and B both designate Pro or a derivative thereof.

89. The peptide according to claim 78, wherein A and B independently represents an amino acid residue having a saturated carbocyclic structure of 4, 5 or 6 members,
wherein said carbocyclic structure further comprises one or more heteroatoms selected from the group consisting of N, O and S.

90. The peptide according to claim 78, wherein B, A¹³, A¹⁴, A¹⁵, and A¹⁶ are absent, and optionally A¹⁰ A, and A¹⁷ are present, or wherein A¹⁰ A, B, A¹³, A¹⁴, A¹⁵, A¹⁶, and A¹⁷ are absent, and optionally A⁸, A⁹, A¹⁸, A¹⁹, A²⁰, and A²¹ are present.

91. The peptide according to claim 78, wherein X is absent and Y and Z are present.

92. A method for reducing or enhancing body weight in a subject, the method comprising administering, to the subject, an appropriately effective amount of (i) a peptide, which is a sequence variant and a functional and/or structural mimic of peptide YY, said peptide comprising at least one modification of the amino acid sequence set forth in SEQ ID NO: 2 (h-PYY 3-36), wherein said peptide
includes a modification that conformationally constrains the relative position of the N-terminal amino acid of that part of SEQ ID NO 2 present in the peptide and amino acid 34 of SEQ ID NO: 2 in the peptide; and/or
includes a branched amino acid sequence resulting in 2 free N-terminal amino acids; and/or
includes N-terminal and/or C-terminal addition of a net basic amino acid sequence;
optionally further includes deletion of amino acids 1-5 of SEQ ID NO: 2; and/or
includes deletion of any one or more of amino acid residues 8-15 of SEQ ID NO: 2 without deletion of all of amino acids 1-7 of SEQ ID NO 2; and/or
includes deletion of amino acids 6 and 7 of SEQ ID NO: 2 without deletion of all of amino acids 1-5 of SEQ ID NO 2; and/or
includes deletion of amino acids 16-19 of SEQ ID NO: 2 without deletion of all of amino acids 1-15 of SEQ ID NO 2; and/or
includes two cross linkable protected Cys amino acid substitutions; wherein said peptide further comprises at most 6 substitutions in the amino acid sequence set forth in SEQ ID NO: 2, each of which is a structure and/or functionality preserving substitution;
or of (ii) a peptide of formula I
R¹-X-Y-Z-A²³-A²⁴-A²⁵-A²⁶-A²⁷-A²⁸-A²⁹-A³⁰-A³¹-A³²-A³³-A³⁴-A³⁵-A³⁶-R² (I)
Wherein
A²² is Ala or a structure and/or functionality preserving substitution thereof;
A²³ is Ser or a structure and/or functionality preserving substitution thereof;
A²⁴ is Leu or a structure and/or functionality preserving substitution thereof, His or Cys;
A²⁵ is Arg or a structure and/or functionality preserving substitution thereof;
A²⁶ is Leu or a structure and/or functionality preserving substitution thereof, His or Cys;
A²⁷ is Tyr or a structure and/or functionality preserving substitution thereof;
A²⁸ is Leu or a structure and/or functionality preserving substitution thereof, or Cys;
A²⁹ is Asn or a structure and/or functionality preserving substitution thereof, or Lys, which is optionally coupled to an amino acid sequence via a peptide bond at the s-amino group;
A³⁰ is Leu or a structure and/or functionality preserving substitution thereof;
A³¹ is Val or a structure and/or functionality preserving substitution thereof, or Cys;
A³² is Thr or a structure and/or functionality preserving substitution thereof;
A³³ is Arg or a structure and/or functionality preserving substitution thereof;
A³⁴ is Gln or a structure and/or functionality preserving substitution thereof;
A³⁵ is Arg or a structure and/or functionality preserving substitution thereof; and
A³⁶ is Tyr or a structure and/or functionality preserving substitution thereof;
Z is a peptide of formula
A¹³-A¹⁴-A¹⁵-A¹⁶-A-¹⁷-A¹⁸-A¹⁹-A²⁰-A²¹
which is absent or wherein,
A¹³ is Ser or a structure and/or functionality preserving substitution thereof or absent;
A¹⁴ is Pro or a structure and/or functionality preserving substitution thereof or absent;
A¹⁵ is Glu or a structure and/or functionality preserving substitution thereof or absent;
A¹⁶ is Glu or a structure and/or functionality preserving substitution thereof or absent;
A¹⁷ is Leu or a structure and/or functionality preserving substitution thereof or absent;
A¹⁸ is Asn or a structure and/or functionality preserving substitution thereof;
A¹⁹ is Arg or a structure and/or functionality preserving substitution thereof;
A²⁰ is Tyr or a structure and/or functionality preserving substitution thereof; and
A²¹ is Tyr or a structure and/or functionality preserving substitution thereof;
Y is a peptide of formula
A⁸-A⁹-A¹⁰-A-B
which is absent or wherein
A⁸ is Pro or a structure and/or functionality preserving substitution thereof;
A⁹ is Gly or a structure and/or functionality preserving substitution thereof,
A¹⁰ is Glu or a structure and/or functionality preserving substitution thereof, or absent; and
A-B designates a dipeptide A¹¹-A¹² selected from the group consisting of Gly-Gly, Pro-Gly, Gly-Pro, Sar-Sar, Sar-Hyp, Hyp-Sar, Pro-Sar, Sar-Pro, Pro-Hyp, Pro-Pro, Hyp-Pro, and Hyp-Hyp, where Pro and Hyp independently may be an L or D form, where the ring structure of Pro and Hyp is optionally substituted with halogen, nitro, methyl, amino, or phenyl, Hyp represents 3-hydroxyproline or 4-hydroxyproline, Sar represents sarcosine, or one or both of the amino acid residues of A-B is a Sar, or an N-cyclohexylglycine residue, or A and B each independently represents a group of the formula II 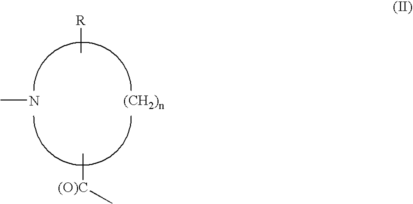
wherein n is an integer having the value 3, 4, or 5, and R represents an optional substituent, preferably selected from the group consisting of halogen, phenyl, hydroxy, NH₂, and C(1-6)alkyl optionally substituted with halogen, or
A-B designates the formula IIa 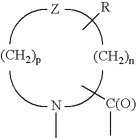
wherein n is an integer having the value 0, 1, 2, and 3, p is an integer having the value 0, 1, 2, and 3, Z represents O or S, and R represents an optional substituent, preferably selected from the group consisting of halogen, phenyl, hydroxy, NH₂, and C(1-6)alkyl, or A and B independently represents an amino acid residue having a saturated carbocyclic structure of 4, 5 or 6 members and where in said carbocyclic structure further comprises one or more heteroatoms,
X is a peptide of formula
A³-A⁴-A⁵-A⁶-A⁷
which is absent or wherein
A³ is Ile or a structure and/or functionality preserving substitution thereof, or Cys;
A⁴ is Lys or a structure and/or functionality preserving substitution thereof;
A⁵ is Pro or a structure and/or functionality preserving substitution thereof, or Cys;
A⁶ is Glu or a structure and/or functionality preserving substitution thereof; and
A⁷ is Ala or a structure and/or functionality preserving substitution thereof, or Cys;
R¹ is absent or an amino acid sequence; and
R² is absent or an amino acid sequence;
wherein said peptide comprises at most one disulfide bridge selected from Cys³-S—S-Cys³¹, Cys³-S—S-CyS²⁸, Cys⁵-S—S-Cys²⁶, and Cys⁷-S—S-Cys²⁴;
or wherein A is absent, Asp or a structure and/or functionality preserving substitution thereof and B is absent, Ala or a structure and/or functionality preserving substitution thereof and said peptide comprises a disulfide bridge selected from Cys³-S—S-cys³¹, Cys³-S—S-Cys²⁸, Cys⁵-S—S-Cys²⁶, and Cys⁷-S—S-Cys²⁴;
wherein the number of structure and/or functionality preserving substitutions does not exceed 6;
wherein the C-terminal amino exposes a free carboxylic acid group or an amide group; and
or a multimer and/or pharmaceutically acceptable salt thereof.
